(19) United States
(12) Patent Application Publication
(10) Pub. No.: US 2026/0279566 A1
(43) Pub. Date: Sep. 17, 2026

(54) Automated Medical Coding or Electronic Health Record Documentation System Agentic AI

(71) Applicants: Bao Q. Tran, Saratoga, CA (US); Khue Duong, Saratoga, CA (US)

(72) Inventors: Bao Q. Tran, Saratoga, CA (US); Khue Duong, Saratoga, CA (US)

(21) Appl. No.: 19/079,774

(22) Filed: Mar. 14, 2025

(51) Int. Cl. G16H 50/20; G06F 9/451; G06F 9/50; G06F 16/34; G06F 40/295; G16H 10/60

(52) U.S. Cl. CPC G16H 50/20; G06F 16/345; G06F 40/295; G16H 10/60; G06F 9/451; G06F 9/5038; G06F 2209/5021

(57) ABSTRACT

Systems and methods are disclosed for automated medical coding by receiving, by a computing system, medical information associated with a patient encounter; analyzing, by the computing system using at least one artificial intelligence model, the medical information to identify clinical concepts present in the medical information; mapping, by the computing system, the identified clinical concepts to one or more standardized medical codes based on at least one coding system; generating, by the computing system, a coding recommendation comprising the one or more standardized medical codes; and storing the one or more standardized medical codes in association with a patient record.

### BACKGROUND OF THE INVENTION

Medical institutions have long relied on manual processes and conventional software for coding medical diagnoses and managing health records, processes that are often labor-intensive, error-prone, and time-consuming. Over the years, the healthcare industry has seen an increasing demand for enhanced accuracy and efficiency in handling large volumes of clinical data, which has led to the exploration and adoption of various computer-assisted approaches. Evolving data complexities, regulatory requirements, and the growth of digital health information have encouraged advancements in the automation of health record documentation and coding tasks. As these challenges have spurred ongoing research and development efforts, the field has gradually shifted toward integrating advanced analytical tools capable of processing nuanced clinical language and diverse data formats to meet the operational demands of modern healthcare settings.

### SUMMARY OF THE INVENTION

In one aspect, a method for automated medical coding comprises receiving medical information linked to a patient encounter via a computing system. In one aspect, the computing system uses one or more artificial intelligence models to analyze the received medical information and identify clinical concepts within it. In one aspect, the identified clinical concepts are mapped to standardized medical codes from a recognized coding system. In one aspect, a coding recommendation that includes these standardized codes is generated by the computing system. In one aspect, this coding recommendation is presented to a user through a user interface, and the standardized medical codes are stored in connection with the patient's record.

In yet another aspect, a computing system receives clinical interaction data associated with a patient encounter. In yet another aspect, the computing system processes the received data using at least one artificial intelligence model to extract clinically relevant information. In yet another aspect, a structured clinical documentation is generated based on the extracted information, and this documentation conforms to predefined documentation standards. In yet another aspect, the structured clinical documentation is presented via a user interface for review by a healthcare provider, who then verifies the documentation. In yet another aspect, the verified structured clinical documentation is stored in an electronic health record database.

In another aspect, a cognitive processing layer of agentic systems leverages advanced NLP capabilities to extract meaningful information from unstructured clinical text. This involves converting clinical narratives into structured, actionable data; identifying medical concepts, diagnoses, procedures, and treatments from free text; recognizing contextual modifiers (negation, uncertainty, temporality); and mapping colloquial terminology to standardized medical vocabulary.

Advantages of one implementation may include one or more of the following. The sheer volume of healthcare data exceeds human processing capacity. Medical knowledge doubles approximately every 73 days according to NIH estimates, making it impossible for human coders to stay current with all relevant information. A 2020 study revealed that improper coding costs the US healthcare system an estimated $262 billion annually, highlighting the inadequacy of human-only approaches. Humans face inherent cognitive limitations when processing complex medical information. Working memory constraints limit the number of coding rules that can be simultaneously considered. Attention fatigue leads to diminished accuracy over time. Context switching between different patients and cases incurs significant cognitive costs. By contrast, AI systems maintain consistent performance levels regardless of task duration or complexity.

Medical coding involves tens of thousands of potential codes in systems like ICD-10 and CPT, with complex interrelationships that must be considered for accurate code assignment. Real-time documentation during patient encounters. Autonomous medical coding solutions have the ability to update coding algorithms and guidelines in real-time. This means that healthcare organizations can stay current with the latest coding standards without manual intervention. This continuous adaptation is simply not possible for human coders, who require formal training and practice for each guideline update.

The system can handle clinical documentation from EHR systems, laboratory results from multiple testing platforms, medical imaging data from PACS systems, real-time patient monitoring data, insurance and billing information, voice transcriptions from patient-provider conversations. The processing pipelines can normalize heterogeneous data formats, resolve conflicts, and create unified patient representations-all in real-time while maintaining data integrity. The sophisticated algorithms and advanced artificial intelligence techniques to analyze patient data and generate accurate medical codes. This processing occurs at a scale where, in one example, the health system's imaging department alone makes 70,000 calls to insurers monthly—a volume impossible for a human to handle.

Agentic AI systems rely on comprehensive medical knowledge graphs and ontologies like UMLS/SNOMED to understand relationships between clinical concepts. The processing involved includes: Maintaining and navigating extensive hierarchical code structures; Applying coding rules that span thousands of pages of guidelines; Resolving code conflicts and exclusions; Determining proper sequencing of diagnosis and procedure codes. Agentic systems can continuously verify coding decisions against regulatory frameworks: Checking compatibility with HIPAA requirements; Validating against CMS coding guidelines; Ensuring compliance with payer-specific policies; Detecting potential fraud, waste, or abuse patterns. This compliance checking occurs in real-time, whereas human auditing typically happens after-the-fact, often days or weeks later.

The processing work required to provide effective agentic AI handling of medical coding and EHR management in real-time involves sophisticated data integration, natural language processing, knowledge graph navigation, chain-of-thought reasoning, and compliance validation-all operating simultaneously at massive scale. The volume, velocity, complexity, and continuous evolution of healthcare information make it impossible for human operators to perform these tasks with comparable efficiency, accuracy, or timeliness, highlighting the necessity of agentic AI systems in modern healthcare operations.

By leveraging one or more artificial intelligence models to parse clinical language and extract relevant concepts, one implementation minimizes human error and subjectivity associated with manual coding. This results in more accurate and standardized mapping of clinical information to recognized medical codes. The automated process streamlines the extraction and documentation of clinical data, significantly reducing the time healthcare providers spend on administrative tasks. This efficiency gain can lessen the burden on staff, allowing them to focus more on patient care.

Collectively, these advantages contribute to improved health information management, reduce burdens associated with traditional manual coding and documentation processes, and create a more robust framework for managing the growing complexities of digital health data.

### BRIEF DESCRIPTION OF DRAWINGS

FIG. 1 shows an exemplary flowchart detailing steps for automated medical coding using AI, from receiving medical information to storing standardized codes.

FIG. 2 shows an exemplary flowchart showing a process to determine and display confidence scores for standardized medical codes.

FIG. 3 shows an exemplary flowchart illustrating the process of receiving user input for coding recommendations and updating an AI model accordingly.

FIG. 4 shows an exemplary flowchart depicting integration of coding recommendations with an EHR system and automatic population of coding fields.

FIG. 5 shows an exemplary flowchart depicting steps in automated medical coding and electronic health record documentation.

FIG. 6 shows an exemplary flowchart with steps for comparing coding recommendations and generating alerts for potential errors.

FIG. 7 shows an exemplary flowchart with steps for analyzing historical coding patterns and customizing coding recommendations.

FIG. 8 shows an exemplary flowchart depicting a process for detecting ambiguities in medical information and generating queries to resolve them.

FIG. 9 shows an exemplary flowchart depicting detection of missing documentation and generating alerts between start and end.

FIG. 10 shows an exemplary flowchart depicting the process of automatically generating and presenting medical documentation for review.

FIG. 11 shows an exemplary flowchart detailing steps for processing clinical interaction data using AI to generate and verify clinical documentation.

FIG. 12 shows an exemplary flowchart showing steps for converting speech to text, identifying clinically relevant entities, and determining relationships between them.

FIG. 13 shows an exemplary flowchart illustrating a process for organizing clinically relevant information into a documentation template, populating it, and generating natural language narratives.

FIG. 14 shows an exemplary flowchart diagram with steps for analyzing clinical information and generating prompts to address gaps.

FIG. 15 shows an illustration of a process for clinical documentation.

FIG. 16 shows an exemplary flowchart showing a process for modifying and updating structured clinical documentation.

FIG. 17 shows an exemplary flowchart with steps for generating and associating medical codes in clinical documentation.

FIG. 18 shows an exemplary flowchart depicting the process of analyzing verified clinical documentation to identify decision support opportunities and presenting recommendations to healthcare providers.

FIG. 19 shows an exemplary flowchart illustrating a process of detecting unaddressed items from a documentation checklist and generating alerts for healthcare providers

FIG. 20 shows an exemplary flowchart illustrating the process of determining context, retrieving historical information, and incorporating it into clinical documentation.

FIG. 21 shows an exemplary flowchart showing the identification and incorporation of social determinants of health into clinical documentation.

FIG. 22 shows an exemplary flowchart showing a process for continuously monitoring, updating, and displaying clinical documentation during a patient encounter.

FIG. 23 shows an illustration of a quality assurance process.

FIG. 24-25 show exemplary agentic AI systems.

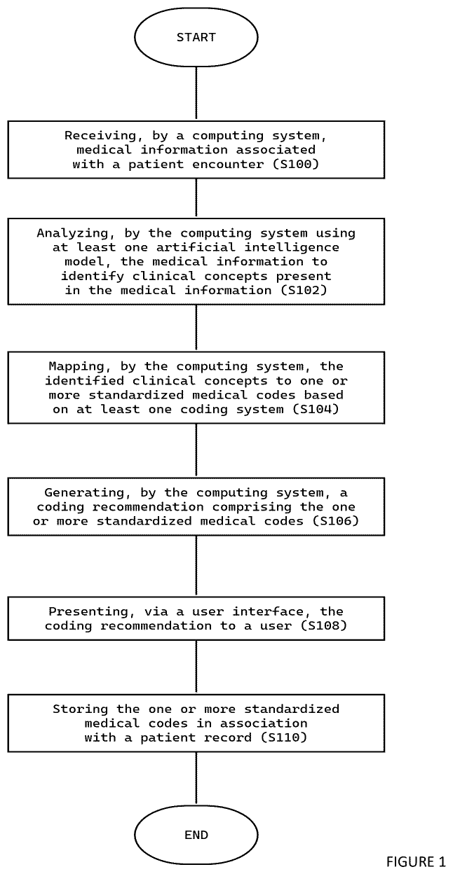

### DETAILED DESCRIPTION OF THE INVENTION

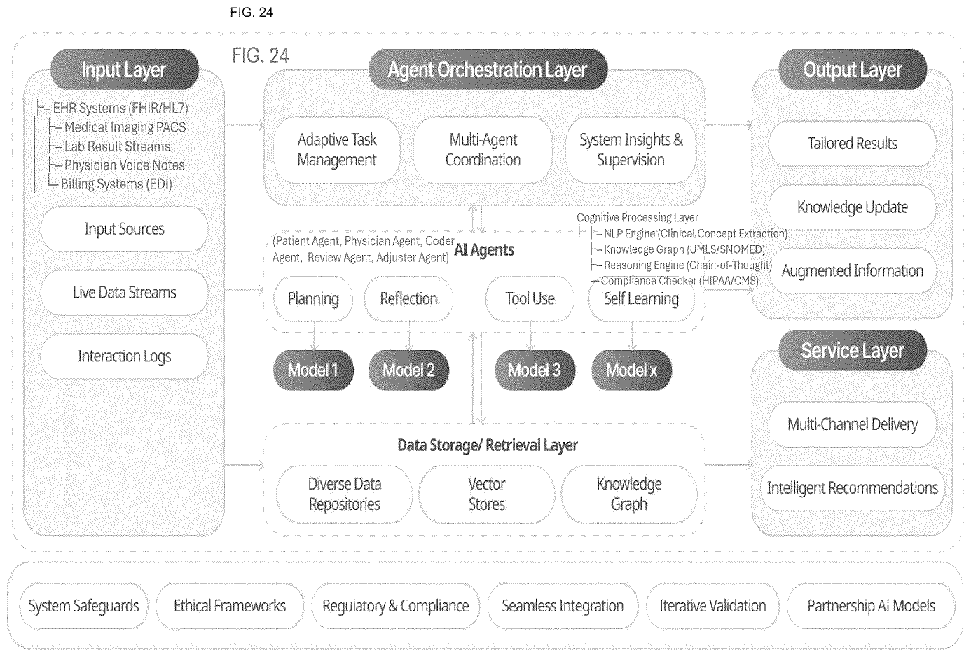

One implementation combines artificial intelligence and real-time data processing to automate medical coding and electronic health record documentation. It processes various forms of medical information-including audio recordings, clinical notes, lab reports, and imaging data—by using an AI model such as a natural language processing model or a fine-tuned large language model to extract clinical concepts, determine relationships among them, and map these to standardized codes like ICD, CPT, HCPCS, and HCC (FIG. 24). Based on this mapping and multimodel agentic AI software, the system produces coding recommendations with confidence scores, displays supporting clinical evidence, and updates patient records in electronic health record systems. Additionally, the system processes data from patient encounters, converting audio, video, or handwritten notes into structured clinical documentation with sections such as the chief complaint, history of present illness, review of systems, physical examination findings, assessment, and plan. This documentation is then reviewed and modified by healthcare providers, with the system detecting gaps, prompting for additional information, and suggesting codes based on the verified content. One implementation also incorporates historical patient data, identifies social determinants of health, accommodates provider-specific preferences, and generates patient-facing summaries in real time.

In one embodiment, the cognitive processing layer of each agent operates as follows. A Cognitive Processing Layer represents the analytical core of modern healthcare AI systems, functioning as the intelligent bridge between raw clinical data and actionable medical insights. This sophisticated component transforms unstructured information into structured, meaningful output through four interconnected elements that collectively mimic human clinical reasoning. The NLP Engine focused on Clinical Concept Extraction serves as the primary interpreter of unstructured medical text, employing sophisticated algorithms to identify and extract clinically relevant entities from diverse documentation. As described in contemporary research, this engine implements a dual-phase process of concept mention detection through named entity recognition (NER) technology, followed by concept encoding that maps identified mentions to standardized terminology. Modern implementations increasingly leverage transformer-based architectures and deep learning approaches, moving beyond traditional rule-based systems to handle the lexical variations and contextual complexities inherent in clinical documentation. Studies have demonstrated this component's effectiveness across multiple medical specialties, with precision ranging from 0.53-0.96 and recall from 0.85-0.99 in extracting entities such as medications, diagnoses, symptoms, and procedures from clinical narratives. Advanced systems like cTAKES (Clinical Text Analysis and Knowledge Extraction System) exemplify this capability, providing frameworks for systematically analyzing unstructured medical text and extracting meaningful clinical concepts.

The Knowledge Graph component, typically leveraging standardized medical ontologies like UMLS (Unified Medical Language System) and SNOMED CT (Systematized Nomenclature of Medicine-Clinical Terms), establishes the semantic foundation necessary for meaningful interpretation of extracted concepts. These comprehensive knowledge structures organize medical terminology into hierarchical relationships, linking concepts through standardized unique identifiers (CUIs) and establishing the semantic bridges essential for understanding clinical context[6]. UMLS serves as a metathesaurus that unifies over 200 different vocabulary sources, while SNOMED CT provides a hierarchical organization of more than 350,000 medical concepts with precise relationships between them. This interconnected knowledge network enables the system to understand that terms like “myocardial infarction,” “heart attack,” and “MI” refer to the same clinical concept, and to recognize that concepts exist within broader medical categories and classifications. The knowledge graph functions as the system's medical “memory,” providing the contextual understanding necessary to interpret vague or ambiguous references in clinical documentation.

The Reasoning Engine implements Chain-of-Thought processing to simulate the logical reasoning patterns employed by healthcare professionals when analyzing patient information. Unlike simple pattern-matching approaches, this component enables multi-hop reasoning across disparate information sources, allowing the system to make inferences that connect patient symptoms to potential diagnoses or treatment considerations. As outlined in recent research developments, this engine employs techniques like probabilistic reasoning, temporal logic, and causal inference to establish logical connections between extracted clinical concepts. For example, when analyzing a patient with documented fatigue, elevated blood glucose, and increased urination, the reasoning engine can follow a logical pathway to suggest diabetes as a potential diagnosis, much as a clinician would. This capability extends beyond simple association to encompass complex clinical reasoning, incorporating medical guidelines, best practices, and contextual factors to arrive at clinically sound conclusions that mirror human diagnostic thinking processes. The Compliance Checker component serves as the regulatory guardian of the system, ensuring all processing adheres to healthcare compliance standards including HIPAA privacy protections and CMS (Centers for Medicare & Medicaid Services) guidelines. This critical safeguard operates continuously throughout the cognitive processing workflow, applying rule-based verification against established regulatory frameworks while monitoring for potential privacy violations or documentation deficiencies[14]. As healthcare AI systems increasingly participate in clinical decision-making, this component verifies that recommendations align with established clinical guidelines, insurance requirements, and documentation standards. The compliance checker implements sophisticated validation algorithms that scan for protected health information (PHI) that might be inappropriately exposed, ensures proper authorization for data access, and validates that documentation meets the necessary standards for procedures, diagnoses, and treatments. This layer of oversight is essential for maintaining regulatory compliance while enabling healthcare organizations to confidently deploy AI-augmented systems in clinical environments where both patient privacy and proper medical documentation are non-negotiable requirements.

The Chain of Thought (CoT) LLM for medical coding integrates clinical knowledge processing and sequential reasoning specifically optimized for accurate code assignment. This specialized module employs a multi-stage reasoning architecture where clinical documentation first passes through a context-aware tokenization process that preserves medical terminology relationships and hierarchies. The system then implements a stepped reasoning pattern by first identifying chief complaints and primary diagnoses, followed by associated conditions and procedures, mirroring how professional coders approach documentation analysis. Each reasoning step is explicitly formulated as internal prompts like “First, I'll identify the principal diagnosis based on admission reason and treatment focus” or “Now, I'll check for any procedures documented that require separate coding.” The architecture incorporates domain-specific attention mechanisms that prioritize clinically significant terms and relationships while maintaining awareness of coding guideline constraints. Unlike general-purpose CoT implementations, the coding variant incorporates specialized verification loops that cross-reference proposed codes against official coding guidelines (such as ICD-10-CM Official Guidelines for Coding and Reporting), ensuring selections align with established sequencing rules, excludes notes, and coding conventions. The module implements backtracking capabilities when conflicting coding rules are encountered, allowing it to reconsider earlier reasoning steps if later discoveries invalidate previous conclusions—for example, when a manifestation code is identified that requires resequencing the principal diagnosis. A time-dependent reasoning handles episodes of care appropriately, distinguishing between initial encounters, subsequent visits, and sequelae. The implementation leverages pre-training on extensive medical coding corpora, including millions of professionally coded records across various specialties, enabling the model to develop nuanced understanding of coding patterns while fine-tuning on carefully curated gold-standard coding examples enhances precision for edge cases. Throughout the reasoning process, the system maintains explicit documentation linkage, creating citations that connect each proposed code to specific evidence in the clinical documentation, thereby establishing an auditable reasoning chain that supports both accurate code assignment and subsequent coding validation.

Unlike conventional automation that follows rigid workflows, agentic AI systems for medical coding operate with contextual awareness, strategic decision-making capabilities, and continuous learning mechanisms. This report examines the operational dynamics of these systems, highlighting how they function in real-world healthcare environments. When a clinical document enters the system, the agent activates with specific objectives:
Maximize Coding Accuracy: The agent works to identify all clinically documented conditions and procedures with precise code assignments.
Ensure Compliant Reimbursement: The agent balances maximizing appropriate reimbursement while maintaining strict compliance with coding guidelines and regulations.
Minimize Human Intervention: The agent aims to resolve coding ambiguities independently, generating physician queries only when necessary and routing to human coders only for cases requiring judgment beyond its capabilities.

Unlike passive coding assistance tools, the agent independently drives the coding process forward, making decisions and taking actions to accomplish these goals. When processing a clinical document, the agent first performs multi-dimensional contextual analysis:
The agent begins by examining the entire clinical encounter context, not just the immediate document. It retrieves patient history from the EHR database, analyzes previous encounters and established diagnoses, and considers specialty-specific patterns. For example, when processing a cardiology progress note, the agent would retrieve the patient's cardiac history, previous procedures, and medication regimen to establish a comprehensive baseline for code selection.

The agent then performs specialized document analysis, examining document structure, clinical narratives, and structured data elements. It distinguishes between different documentation components (chief complaint, history of present illness, examination findings, assessment and plan) and applies appropriate analysis strategies to each section. The agent recognizes that information in the assessment section carries different weight than similar information mentioned in the history section.

Following contextual analysis, the agent performs sophisticated clinical concept extraction. The agent identifies both explicit and implicit clinical concepts using advanced natural language processing. Beyond simple keyword matching, it recognizes clinical concepts expressed in various ways. For instance, when encountering text like “the patient's breathing has worsened with increased use of rescue inhaler,” the agent identifies this as representing a potential asthma exacerbation.

The agent then maps relationships between extracted concepts, creating a semantic network of clinical information. It distinguishes between causal relationships (condition A causing condition B), temporal relationships (condition preceding treatment), and contextual qualifiers (severity, acuity, laterality). This relationship mapping is critical for accurate code assignment, as it helps distinguish primary diagnoses from secondary conditions and complications:
a. When selecting diagnosis codes, the agent implements a multi-step process:
b. It first identifies the primary reason for the encounter
c. Maps the condition to an initial code candidate set
d. Applies specificity refinement by searching for clinical details that support more specific code assignment
e. Evaluates the clinical significance of each potential comorbidity to determine inclusion

For example, when coding a diabetic patient with documented peripheral neuropathy who presents for pneumonia, the agent would:
a. Identify pneumonia as the primary reason for the encounter
b. Select appropriate pneumonia code based on documented organism and severity
c. Include the diabetes with peripheral neuropathy as a relevant comorbidity
d. Ensure the diabetes code includes the appropriate manifestation linkage

The agent then performs code optimization, evaluating potential code combinations against compliance guidelines, hierarchical coding rules, and reimbursement impact. Importantly, this optimization occurs within strict compliance boundaries—the agent will not suggest codes not supported by documentation, but will identify the most appropriate combination of supported codes.

A critical operational aspect of agentic AI in medical coding is its ability to make confidence-based routing decisions:
a. For each coding decision, the agent calculates multi-dimensional confidence scores that consider:
b. Documentation clarity and completeness
c. Semantic match between clinical text and code definitions
d. Historical coding patterns for similar cases
e. Potential compliance risks

These scores determine the subsequent workflow. High-confidence cases proceed to automated finalization, moderate-confidence cases receive targeted human review of specific codes, and low-confidence cases route to full human review.

For example, a straightforward case of strep pharyngitis with clear documentation might receive a 95% confidence score and proceed automatically. A complex case involving a surgical complication with ambiguous documentation might receive a 60% confidence score and route for human review with specific questions highlighted for the reviewer.

The agent employs sophisticated decision logic when determining whether to generate physician queries. The agent analyzes documentation gaps against specific criteria:
a. Clinical significance of the missing information
b. Impact on code assignment and reimbursement
c. Likelihood that the physician possesses the missing information
d. Query compliance with regulatory guidelines
e. Based on this analysis, the agent may:
f. Generate a compliant physician query
g. Proceed with coding based on available documentation
h. Flag the case for coding specialist intervention

For example, if documentation indicates “renal insufficiency” without specifying acuity or stage, the agent would recognize this as insufficient for specific code assignment, calculate the reimbursement impact, and generate an appropriate physician query.

Throughout its operations, the agent continuously performs compliance risk assessment. For each coding decision, the agent evaluates:
a. Alignment with Official Coding Guidelines
b. Consistency with National Correct Coding Initiative (NCCI) edits
c. Adherence to payer-specific requirements
d. Risk of triggering audit flags
e. When identifying potential compliance risks, the agent may:
f. Apply more conservative coding approaches for high-risk areas
g. Generate detailed compliance notes for human review
h. Flag specific codes or code combinations for validation

For instance, when coding a procedure with a history of high audit focus (e.g., certain cardiac interventions), the agent would apply heightened scrutiny to documentation requirements and potentially route for dedicated compliance review even if confidence is otherwise high.

Agentic AI coding systems continuously improve through sophisticated feedback incorporation. When human coders modify agent-suggested codes, the agent:
a. Identifies specific points of disagreement
b. Analyzes the patterns and rationale behind human corrections
c. Updates its internal models to reduce similar discrepancies
d. Adjusts confidence thresholds for similar future cases
e. This process operates at multiple levels:
f. Individual concept-to-code mapping adjustments
g. Document pattern recognition refinement
h. Specialty-specific coding pattern updates
i. User-specific preference learning

For example, if a hospital's coding team consistently applies a particular approach to coding certain respiratory conditions that differs slightly from the agent's initial approach, the system will progressively align with the local coding patterns while maintaining compliance.

The agent continuously monitors for: ICD-10-CM/PCS annual updates, CPT code changes and guidance updates, Regulatory bulletins and compliance advisories, and Payer policy updates. When detecting guideline changes, the agent: Analyzes the scope and impact of the changes, Updates internal coding rules and validation checks, Adjusts code relationship maps and hierarchy models, Identifies previously coded cases that might require review. For instance, when ICD-10-CM introduces new codes for a previously less specific condition (as occurred with COVID-19 related conditions), the agent would update its concept mapping tables, modify its specificity requirements, and potentially flag recent cases with the outdated codes for review.

For example, in an integrated environment, when a physician completes a progress note, the agent would immediately analyze it, potentially generate a query visible directly in the EHR, and make the coding available to both billing systems and quality reporting modules. If certain code combinations consistently trigger payer denials, the feedback loop would adjust the agent's code selection strategies to reduce similar denials in the future while maintaining clinical accuracy.

Agentic coding systems implement sophisticated work distribution between AI and human coders. The agent dynamically allocates coding tasks based on: Case complexity and ambiguity scores, Coder expertise and specialization, AI confidence in specific coding domains, Regulatory risk assessment. For example, a multispecialty clinic might configure the system to fully automate routine primary care visit coding, provide coding recommendations for specialist visits requiring human verification, and offer decision support for complex surgical cases while leaving final code selection to specialized human coders. A hospital implementing the system might initially set conservative thresholds requiring human review for most codes, then gradually increase automation levels for specific departments as performance data confirms reliable coding accuracy. When coding a complex procedure with strict documentation requirements, the agent would verify that all required elements are documented, that appropriate medical necessity is established, and that code combinations don't violate bundling rules. If accuracy analysis reveals challenges with a particular specialty or condition type, the system would flag this area for focused improvement, potentially adjusting confidence thresholds, enhancing specialty-specific models, or recommending additional training. The operational dynamics of agentic AI in medical coding represent a fundamental shift from traditional automation approaches. By combining contextual awareness, strategic decision-making, continuous learning, and seamless integration with healthcare systems, these agents function as autonomous coding specialists rather than simple tools. This transformation addresses long-standing challenges in the medical coding domain-reducing the administrative burden on healthcare organizations while improving coding accuracy, compliance, and appropriate reimbursement. As these systems continue to evolve, they promise to further enhance the efficiency and effectiveness of healthcare revenue cycle operations, ultimately supporting the broader goals of healthcare delivery.

Reference label S100 pertains to the initial step in the system in which a computing system receives medical information associated with a patient encounter. This step involves the automated collection and input of various types of clinical data, sourced from electronic health records or other patient care-related sources. By obtaining this information, the computing system establishes the basis for further processing and analysis using advanced artificial intelligence techniques. The reception of medical information under S100 is essential as it ensures that the system has the necessary contextual data to accurately execute subsequent tasks such as identifying clinical concepts and mapping them to standardized medical codes.

The computing system maps the identified clinical concepts to one or more standardized medical codes as defined by reference label S104. In this step, the computing system takes each clinical concept extracted from the medical information and determines the corresponding standardized code based on at least one recognized coding system. This mapping task establishes a systematic association between the extracted clinical concepts and the standardized codes, ensuring that each clinical concept is accurately and consistently represented in a coded format for subsequent processing and storage.

The computing system produces a coding recommendation that comprises one or more standardized medical codes based on the clinical concepts identified within the medical information. This generation step is performed after the system has analyzed the patient encounter data using one or more artificial intelligence models and has mapped the extracted clinical concepts to codes drawn from a recognized coding system. The coding recommendation is designed to offer a precise and consistent representation of the patient encounter through the incorporation of these standardized codes. The process ensures that the output is readily integrable with subsequent healthcare workflows, contributing to greater accuracy in clinical documentation and subsequent record keeping.

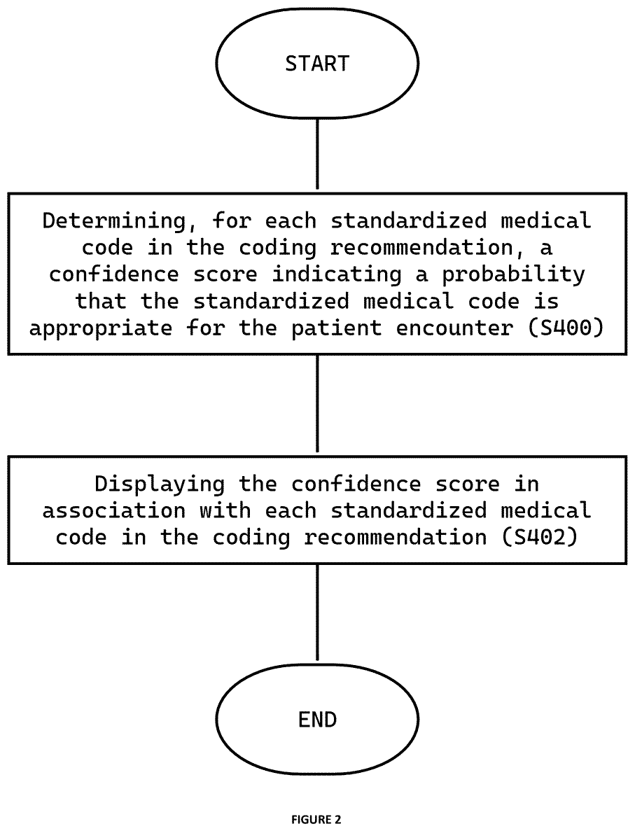

FIG. 2 illustrates a process flowchart for determining and displaying confidence scores for standardized medical codes.

The process involves determining a confidence score for each standardized medical code included in the coding recommendation. This score reflects the likelihood that the code is suitable for the specific patient encounter.

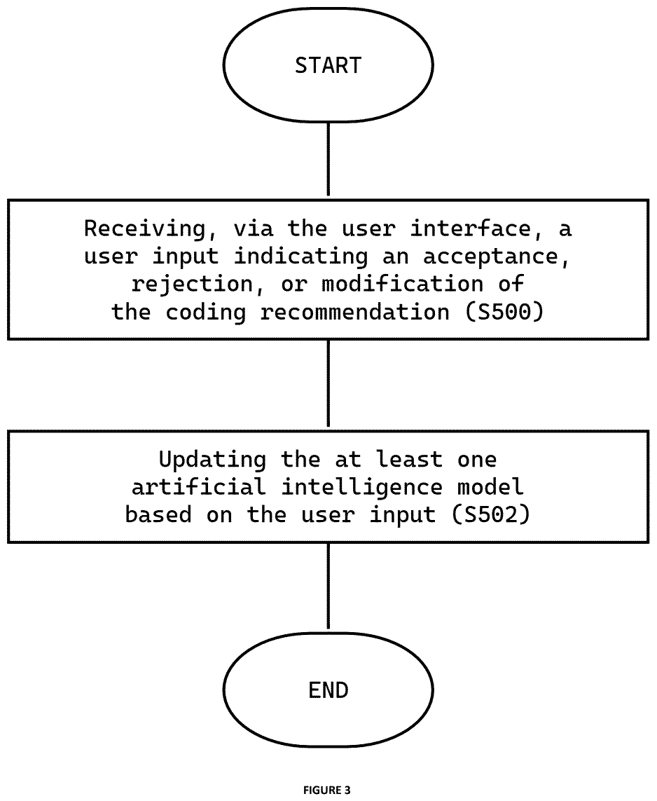

FIG. 3 illustrates the flowchart for receiving user input on coding recommendations and updating an AI model accordingly.

The flowchart illustrates the step of receiving user input via a user interface, which signifies acceptance, rejection, or modification of a coding recommendation. This step is labeled with reference sign S500.

In process step S502, the computing system updates at least one artificial intelligence model based on user input. This input, received via a user interface, indicates acceptance, rejection, or modification of the coding recommendation previously provided by the system. The update process refines and enhances the model's accuracy in generating future coding recommendations by incorporating feedback from user interactions, thereby continually improving the system's performance in clinical documentation and medical coding contexts.

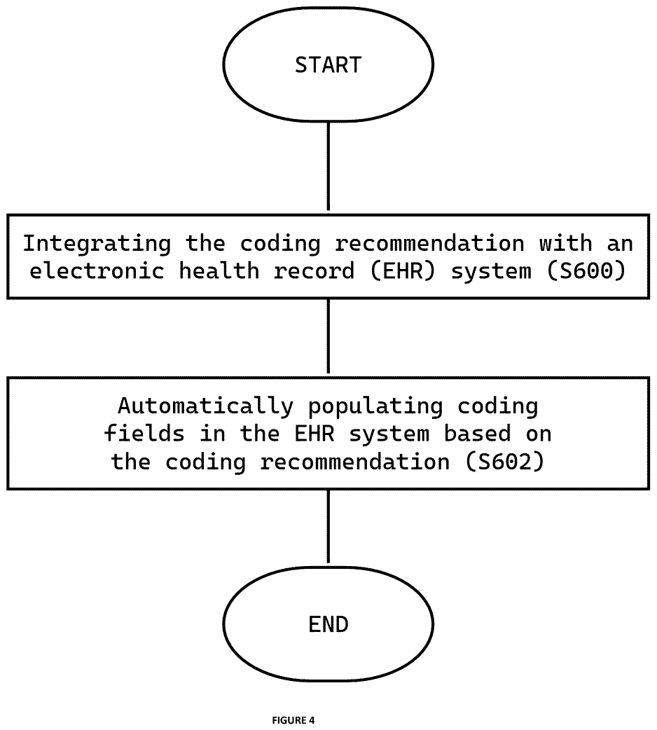

FIG. 4 illustrates the process of integrating coding recommendations with an electronic health record (EHR) system and the automatic population of coding fields.

The flowchart illustrates the process where a computing system integrates the coding recommendation with an electronic health record (EHR) system, as indicated by reference label S600. This integration ensures that the coding recommendation is seamlessly incorporated into the EHR system, allowing for streamlined access and management of patient records.

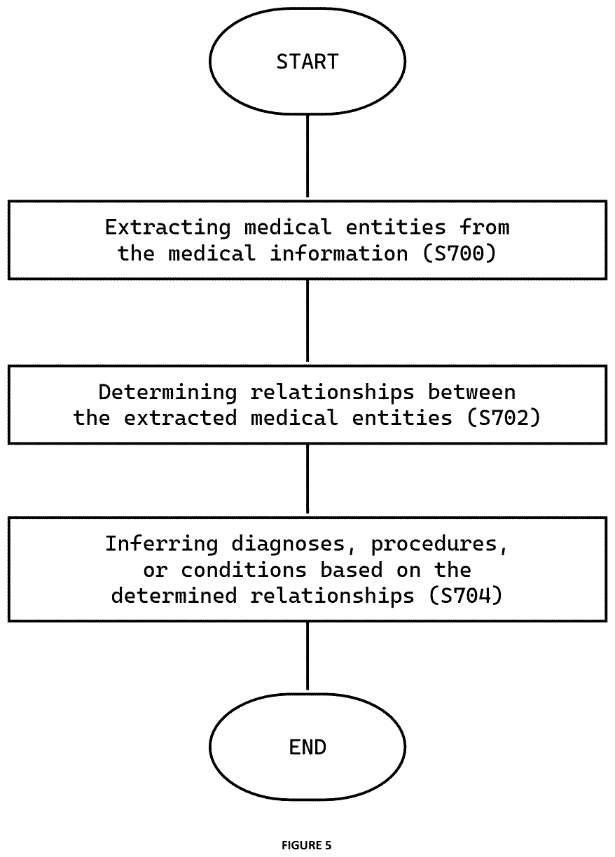

FIG. 5 illustrates the steps involved in automated medical coding and electronic health record documentation.

The flowchart depicted in FIG. 5 illustrates the process of extracting medical entities from the medical information. In this step, labeled S700, a computing system analyzes patient encounter data to identify key medical concepts. This is a foundational element that forms the basis for subsequent analysis and coding procedures, where extracted entities are used to determine relationships and infer diagnoses, procedures, or conditions relevant to the patient's care.

The reference label “determining relationships between the extracted medical entities S702” signifies a step in the method where the computing system, after extracting medical entities from the information, analyzes these entities to establish connections or associations between them. This analysis enables the system to understand how different medical entities interact or relate to one another, forming the basis for subsequent inferences such as diagnoses, procedures, or conditions that are essential for accurate medical coding and documentation.

The reference label “inferring diagnoses, procedures, or conditions based on the determined relationships (S704)” pertains to a specific process within a method for automated medical coding. This step involves the use of a computing system to infer potential diagnoses, procedures, or medical conditions. It does so by analyzing and interpreting the relationships between extracted medical entities from the received medical information. The aim is to enhance the accuracy of medical coding by utilizing artificial intelligence to identify clinically relevant insights from the data.

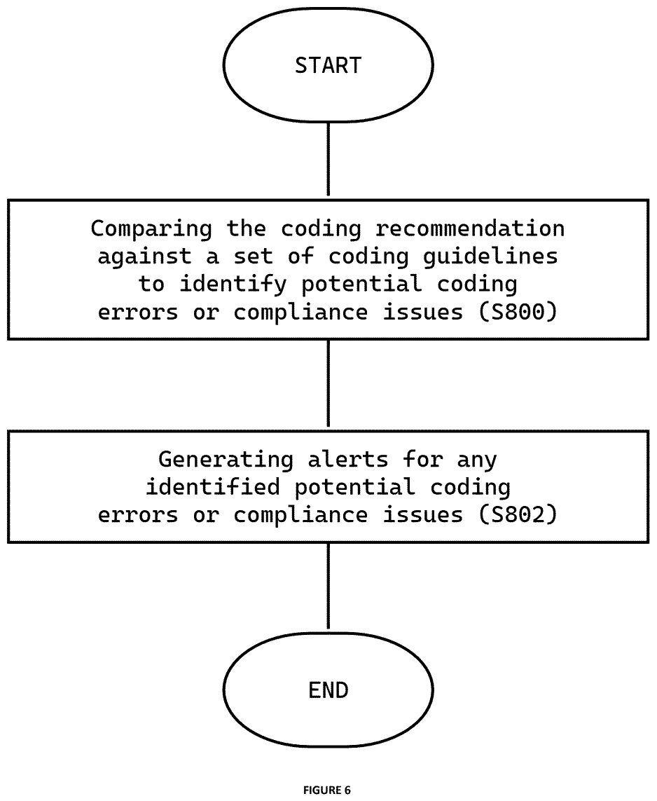

FIG. 6 illustrates the process of comparing coding recommendations against a set of guidelines to identify potential errors and compliance issues, followed by generating alerts for any identified issues.

The computing system compares the coding recommendation to a predefined set of coding guidelines to detect potential coding errors or compliance issues. This process includes an analytical assessment to ensure that the coding conforms to established standards and to identify discrepancies that occur during the process.

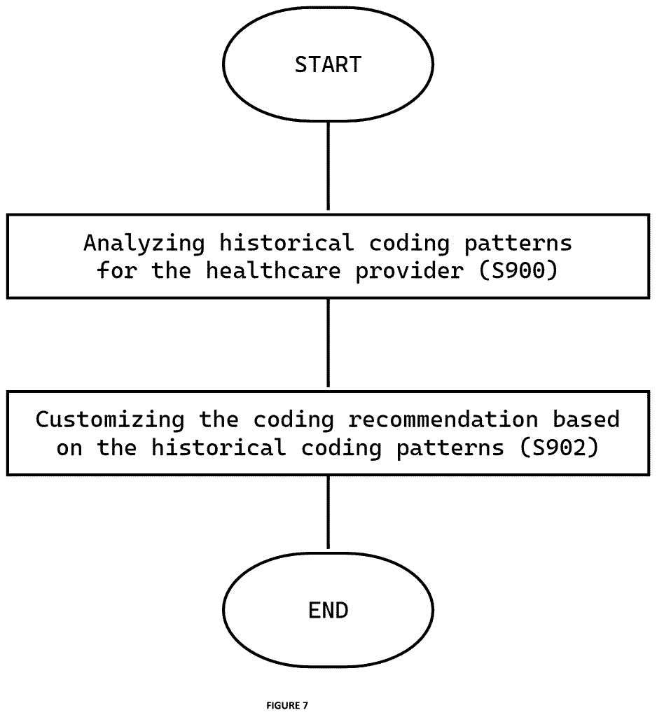

FIG. 7 illustrates the steps involved in analyzing historical coding patterns and customizing coding recommendations.

The step labeled “S900” involves analyzing the historical coding patterns utilized by a healthcare provider. This analysis is intended to understand past coding behaviors and tendencies, which can then be leveraged to make informed adjustments to current coding recommendations. By examining these patterns, the computing system can identify trends and common practices, enabling it to tailor future coding suggestions more precisely to align with the provider's established methodologies.

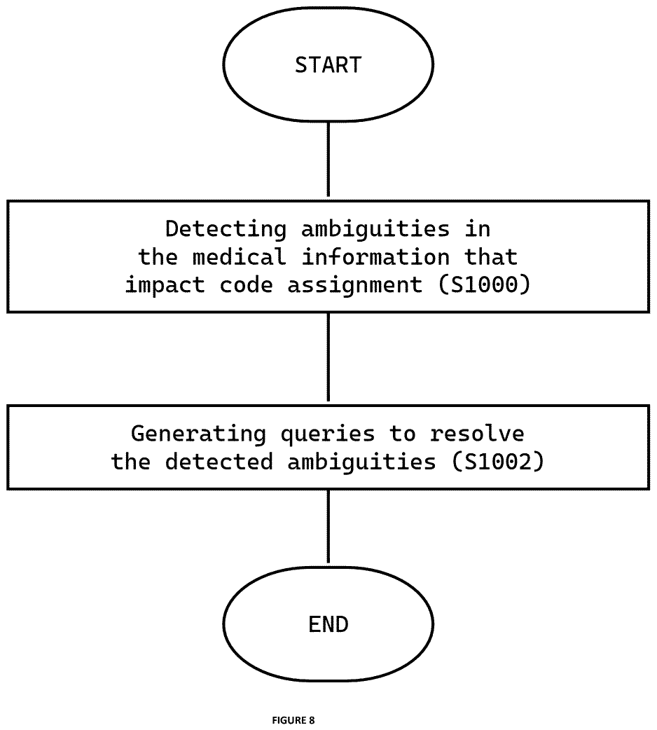

FIG. 8 illustrates a flowchart depicting a process for detecting ambiguities in medical information that impact code assignment and generating queries to resolve them.

The reference label “S1002” pertains to the process of generating queries designed to resolve detected ambiguities within medical information that can affect code assignment. This step follows the detection phase, during which any uncertainties in the medical information are recognized as potentially influencing the correctness or appropriateness of the coding process. Generating these queries enables clarification and ensures more accurate and reliable medical coding outcomes.

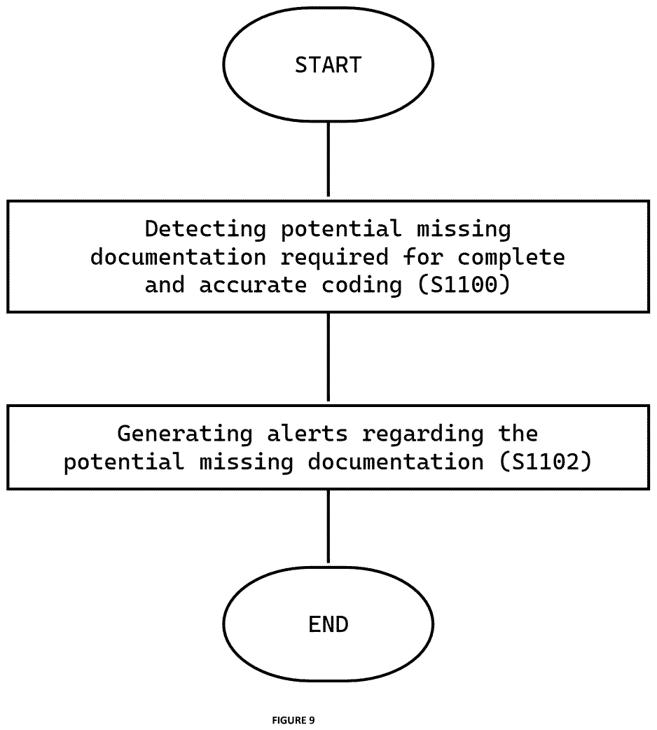

FIG. 9 illustrates the process of detecting missing documentation and generating alerts, beginning with the detection of potential missing documentation required for complete and accurate coding, and ending with the generation of alerts regarding the missing information.

In the depicted process flow, the step labeled “detecting potential missing documentation required for complete and accurate coding” (S1100) is an integral component. This step entails reviewing the available information from a patient encounter to pinpoint any gaps or missing details that might impede the assignment of precise and comprehensive medical codes. Its objective is to ensure that all required documentation is present for effective medical coding, thereby sustaining the accuracy of healthcare records.

The reference label “S1102” pertains to the generation of alerts regarding potential missing documentation required for complete and accurate coding. This step occurs after the detection of missing documentation, as part of a process designed to ensure thorough and precise documentation in medical coding.

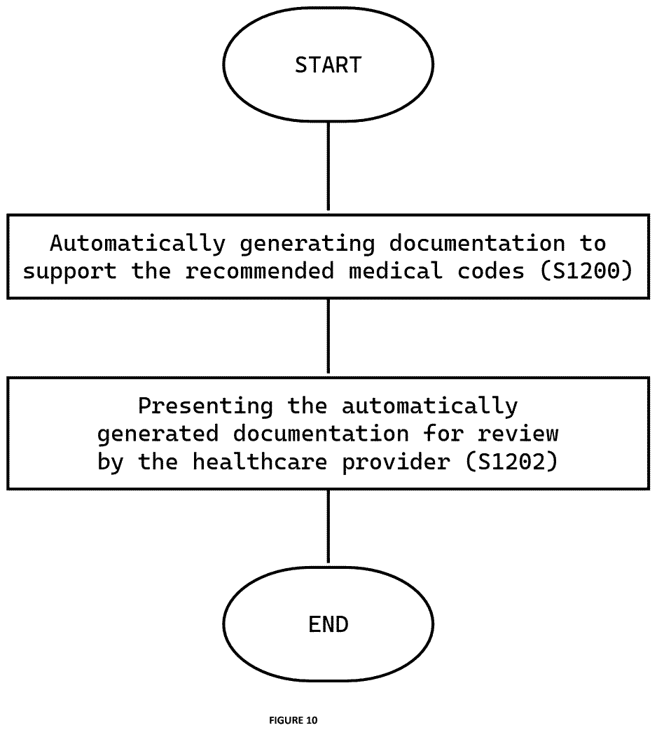

FIG. 10 illustrates the process of automatically generating and presenting medical documentation for review.

The process commences with the computing system automatically generating documentation to support the recommended medical codes. Utilizing sophisticated algorithms, this function ensures that the documentation precisely reflects and substantiates the suggested codes. Once generated, this documentation is prepared for presentation to the healthcare provider, enabling a thorough review process.

The reference label “presenting the automatically generated documentation for review by the healthcare provider (S1202)” refers to a step in the flowchart where the system displays the documentation that has been automatically created. This documentation corresponds to the recommended medical codes and is shown to the healthcare provider for their evaluation and potential confirmation, ensuring it aligns with the clinical context and requirements.

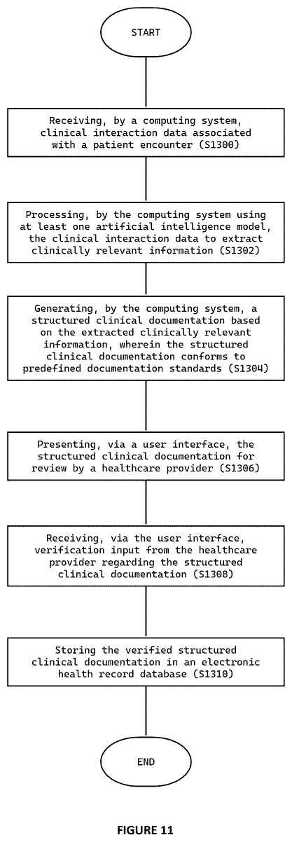

FIG. 11 illustrates a flowchart detailing the steps for processing clinical interaction data using AI. This includes generating and verifying clinical documentation, starting with the reception of data and concluding with its storage in an electronic health record database.

The method commences with the reception of clinical interaction data linked to a patient encounter by a computing system (S1300). This step involves collecting detailed information generated during healthcare interactions, which forms the basis for further processing and analysis.

The computing system processes clinical interaction data by employing at least one artificial intelligence model to extract clinically relevant information. This extraction is essential for ensuring that vital details from the patient encounter are accurately captured and utilized in subsequent documentation and coding processes.

In an aspect of one implementation, the computing system presents structured clinical documentation via a user interface. This allows a healthcare provider to review the documentation, ensuring that it aligns with predefined standards and accurately reflects the extracted clinically relevant information from the patient encounter.

In the described system, once the structured clinical documentation is reviewed and verified by a healthcare provider via a user interface, it is subsequently stored in an electronic health record database. This step ensures that the verified documentation is securely archived for future reference and accessibility, finalized by the operation labeled as S1310.

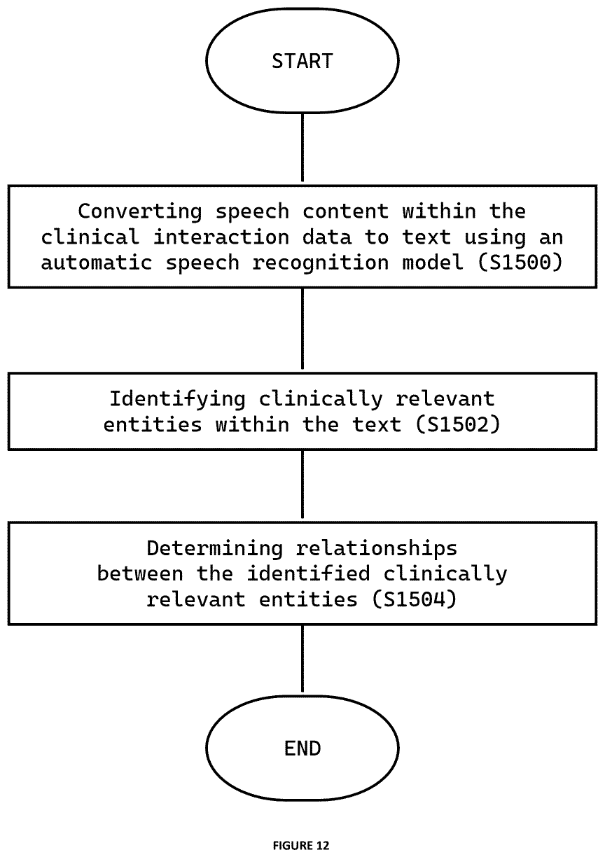

FIG. 12 illustrates the process flow for converting speech to text, identifying clinically relevant entities, and determining relationships between them.

The reference label “converting speech content within the clinical interaction data to text using an automatic speech recognition model” (S1500) pertains to a step where spoken words from clinical interactions are transcribed into written text. This process utilizes an automatic speech recognition (ASR) model, which is designed to accurately capture and convert speech to a textual format. This conversion is essential for further processing and analysis of clinical data.

In step S1502, the method involves identifying clinically relevant entities within the text. This process occurs after converting the speech content from the clinical interaction data into text. The goal is to pinpoint specific medical terms, conditions, or elements that are essential for understanding the clinical encounter and contextualizing the patient's medical information.

In the context of the flowchart, “determining relationships between the identified clinically relevant entities (S1504)” refers to the process where the system analyzes the identified entities within the converted text to understand how they are interconnected. This involves examining correlations or interactions among medical terms or data points, providing insights essential for accurate diagnosis and treatment planning.

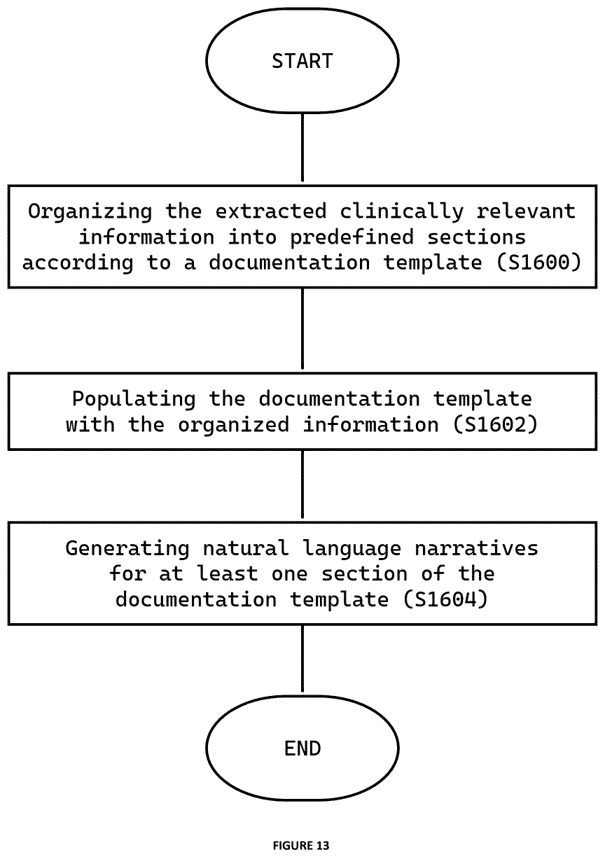

FIG. 13 illustrates the process of organizing clinically relevant information into a documentation template, then populating it and generating natural language narratives.

The computing system organizes the extracted clinically relevant information into predefined sections in accordance with a documentation template. This step involves structuring the information systematically to ensure that it aligns with standardized documentation formats, facilitating easy review and comprehension.

The step labeled “S1604” involves the generation of natural language narratives for at least one section of the documentation template. This process transforms the organized information into narrative form, enhancing readability and comprehension for healthcare providers reviewing the documentation.

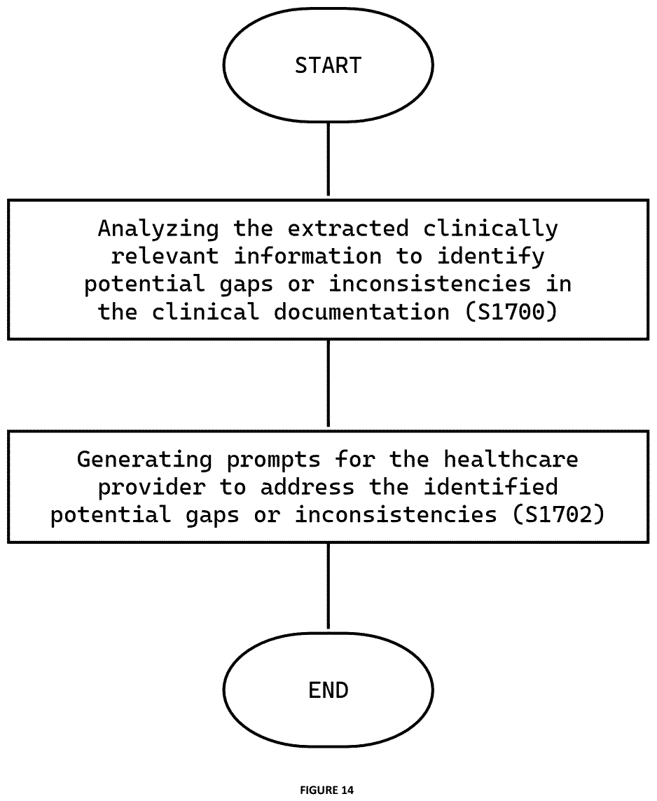

FIG. 14 illustrates a flowchart diagram detailing the steps for analyzing clinical information and generating prompts to address gaps.

The reference label “S1700” pertains to a method for analyzing extracted clinically relevant information to pinpoint potential gaps or inconsistencies within clinical documentation. This step involves systematically reviewing the data to identify any missing elements or contradictions that impact the accuracy and completeness of the records.

The reference label “generating prompts for the healthcare provider to address the identified potential gaps or inconsistencies S1702” pertains to a process where, after analyzing clinically relevant information, the system identifies any potential gaps or inconsistencies in the clinical documentation. Subsequently, prompts are generated for the healthcare provider, guiding them to address these identified issues. This step aims to enhance the completeness and accuracy of the medical records by ensuring that all pertinent aspects are thoroughly documented and any possible errors or omissions are rectified.

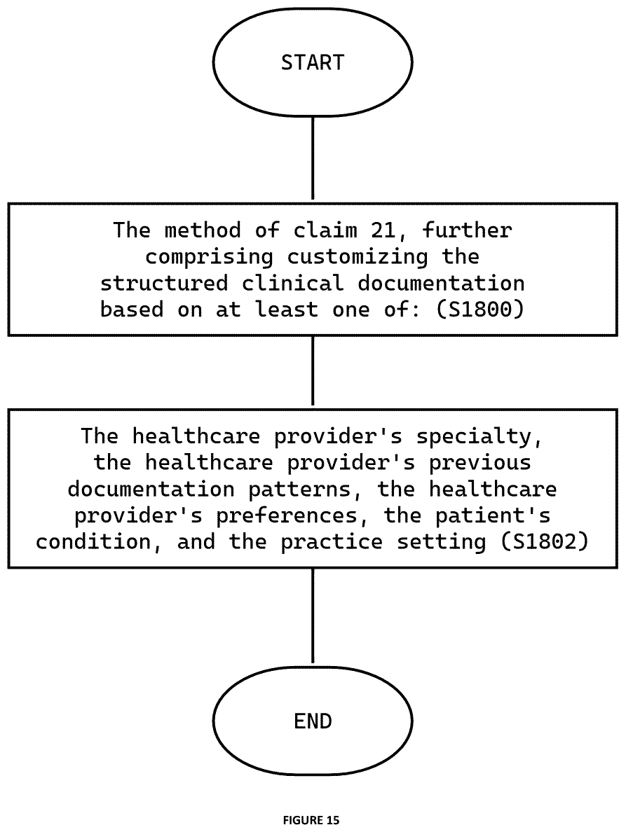

FIG. 15 illustrates the customization process of structured clinical documentation, based on factors such as the healthcare provider's specialty, previous documentation patterns, preferences, the patient's condition, and the practice setting.

The reference label “S1800” pertains to the customization of structured clinical documentation within a patient encounter process. This customization takes into account factors such as the healthcare provider's specialty, previous documentation patterns, preferences, the patient's condition, and the practice setting. This ensures that the documentation aligns closely with specific contextual needs and enhances accuracy and relevance in the medical coding process.

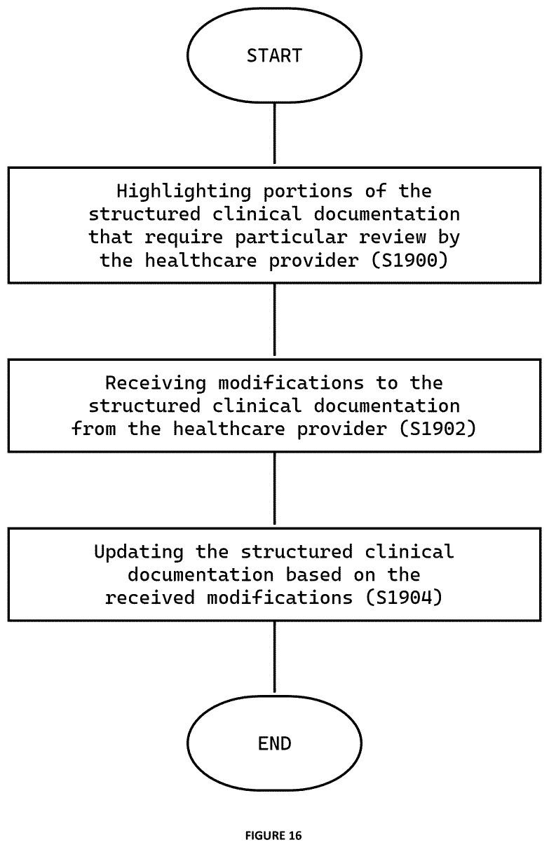

FIG. 16 illustrates a process for modifying and updating structured clinical documentation. The flowchart details steps including highlighting portions requiring review, receiving modifications from healthcare providers, and updating the documentation accordingly.

The process begins by marking specific sections of the structured clinical documentation that call for a detailed review by the healthcare provider. This step ensures that all pertinent aspects of the documentation receive the necessary focus, thereby supporting an accurate and comprehensive evaluation.

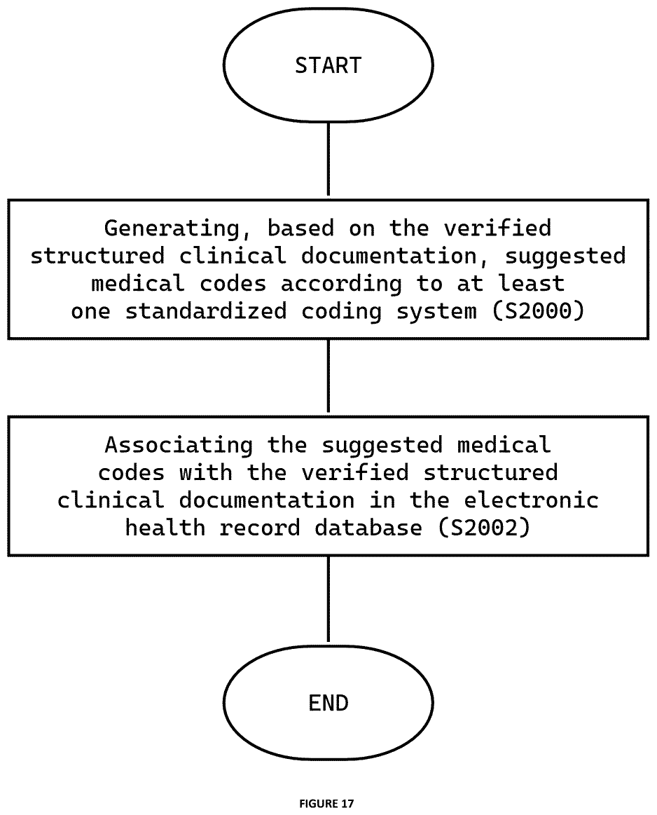

FIG. 17 illustrates the process for generating and associating medical codes in clinical documentation, using a flowchart that outlines the steps involved. The reference label “associating the suggested medical codes with the verified structured clinical documentation in the electronic health record database S2002” represents a process where the computing system links the recommended medical codes to the confirmed clinical documentation within an electronic health record (EHR) database. This step ensures that the structured documentation, which has been verified for accuracy, is integrated with corresponding coding information, thereby maintaining consistent and comprehensive medical records within the EHR system.

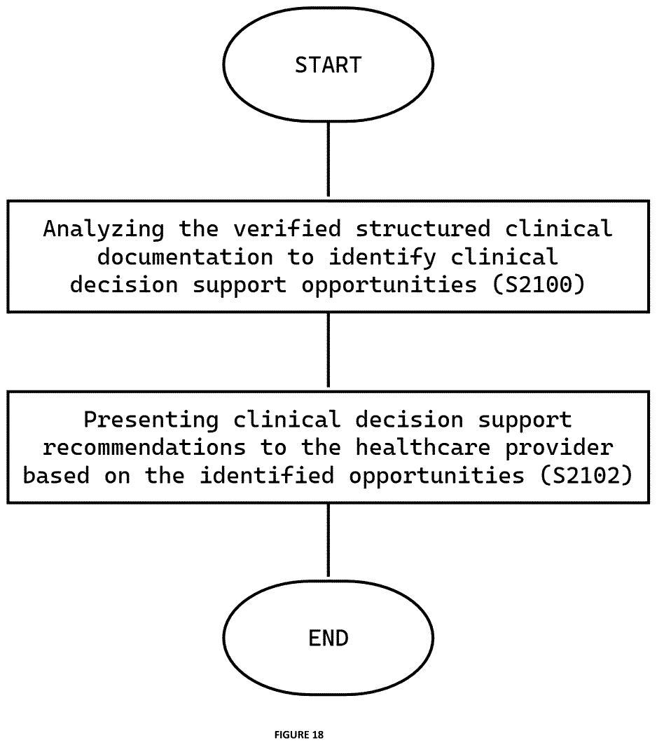

FIG. 18 illustrates the process of analyzing verified clinical documentation to identify decision support opportunities and presenting recommendations to healthcare providers.

The reference label “S2100” refers to the step of analyzing verified structured clinical documentation with the objective of identifying clinical decision support opportunities. In this process, the computing system evaluates the documentation to uncover potential interventions or recommendations that could assist healthcare providers in making informed decisions regarding patient care.

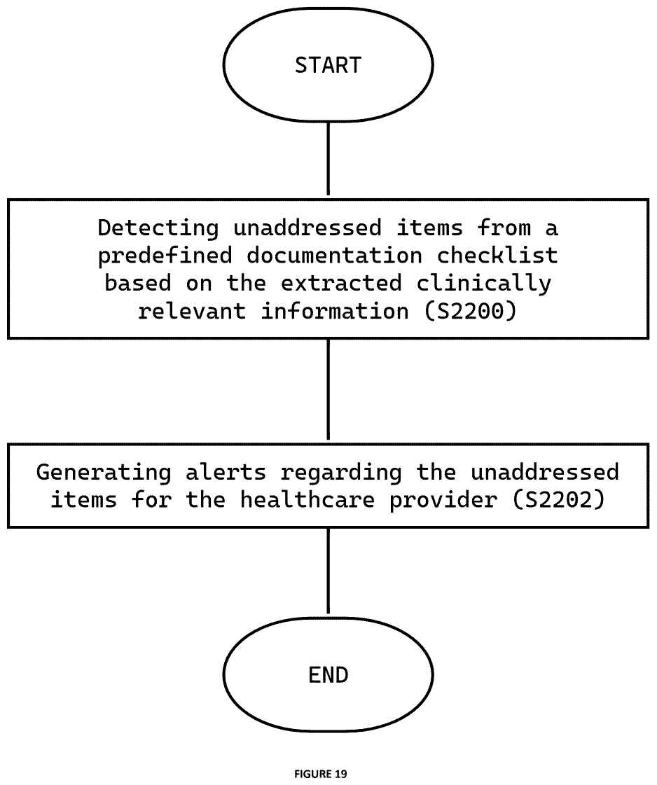

FIG. 19 illustrates the process of detecting unaddressed items from a predefined documentation checklist, based on extracted clinically relevant information, and generating alerts for healthcare providers.

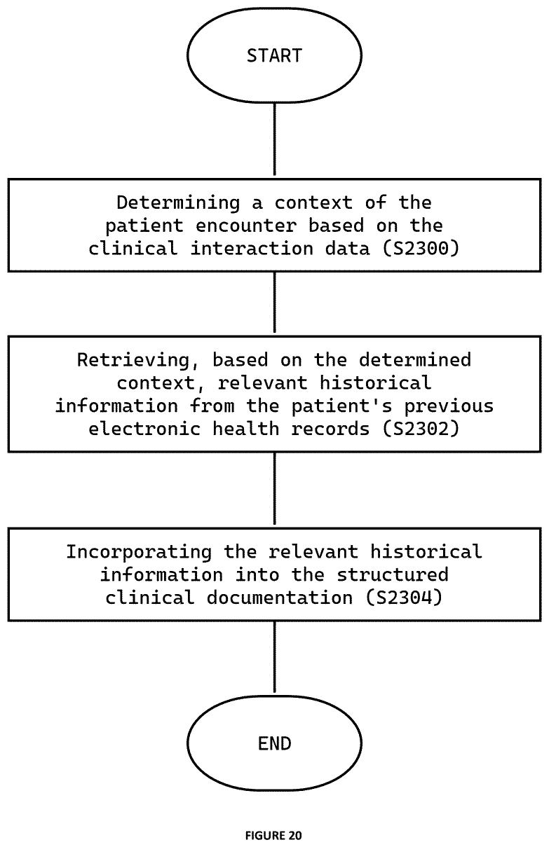

FIG. 20 illustrates the flowchart that demonstrates the process of determining the context of a patient encounter, retrieving relevant historical information from electronic health records, and incorporating that data into structured clinical documentation.

In one aspect, the method involves determining a context of the patient encounter based on clinical interaction data. This step is essential for understanding the specific circumstances surrounding the medical visit, including the patient's current health status and any relevant details discussed during the encounter. By analyzing this data, the system can appropriately tailor subsequent actions, such as retrieving pertinent historical information from the patient's previous electronic health records.

In the context of this one implementation, step S2304 involves incorporating relevant historical information into structured clinical documentation. This step follows the determination of the patient encounter's context and retrieval of pertinent data from previous electronic health records. It ensures that the patient's historical medical information is seamlessly integrated into current documentation, providing a comprehensive view for healthcare providers.

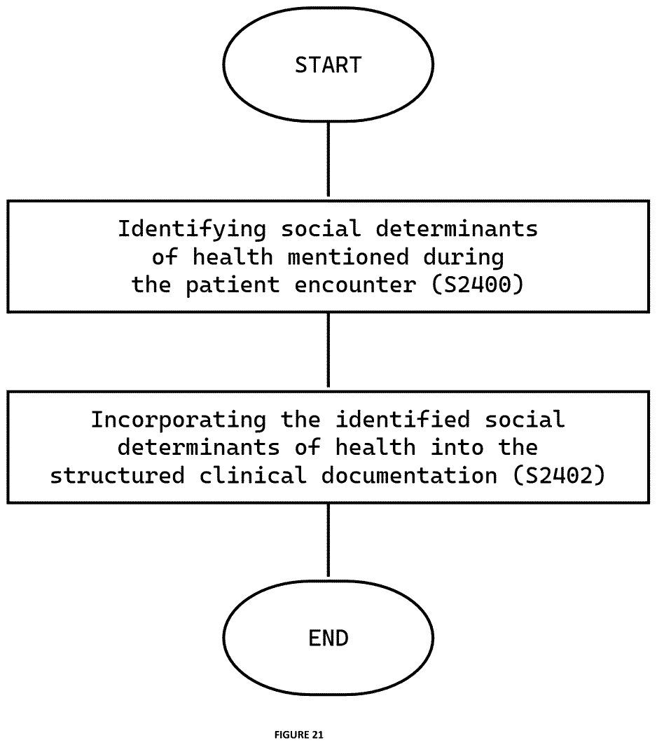

FIG. 21 illustrates the process of identifying and incorporating social determinants of health into clinical documentation. The reference label “S2400” pertains to the process within a computing system where social determinants of health mentioned during a patient encounter are identified. This step involves recognizing and extracting relevant social factors that could influence a patient's health. These determinants are then noted for further incorporation into the structured clinical documentation, ensuring a comprehensive record that accounts for both medical and social aspects impacting the patient's health.

In one embodiment, a computing system incorporates identified social determinants of health into structured clinical documentation. This integration occurs following the identification of these determinants during a patient encounter, enhancing the contextual relevance and comprehensiveness of the clinical records. This approach ensures that the social factors influencing patient health are systematically documented and considered in clinical decision-making processes.

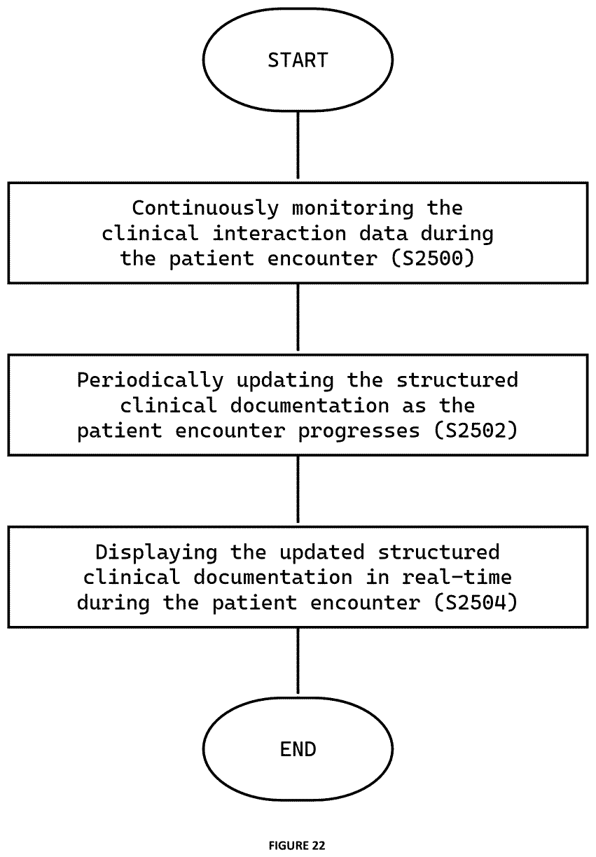

FIG. 22 illustrates the following references: continuously monitoring the clinical interaction data during a patient encounter, periodically updating the structured clinical documentation as the encounter progresses, and displaying the updated documentation in real time.

The label “continuously monitoring the clinical interaction data during the patient encounter S2500” refers to the process by which a computing system persistently observes and logs data throughout a patient's visit. This ensures that all interactions, whether spoken or recorded in other formats, are captured in real-time. The continuous monitoring facilitates an ongoing update of relevant information as the encounter progresses, enabling accurate and up-to-date documentation that reflects the dynamic nature of the patient-provider interaction.

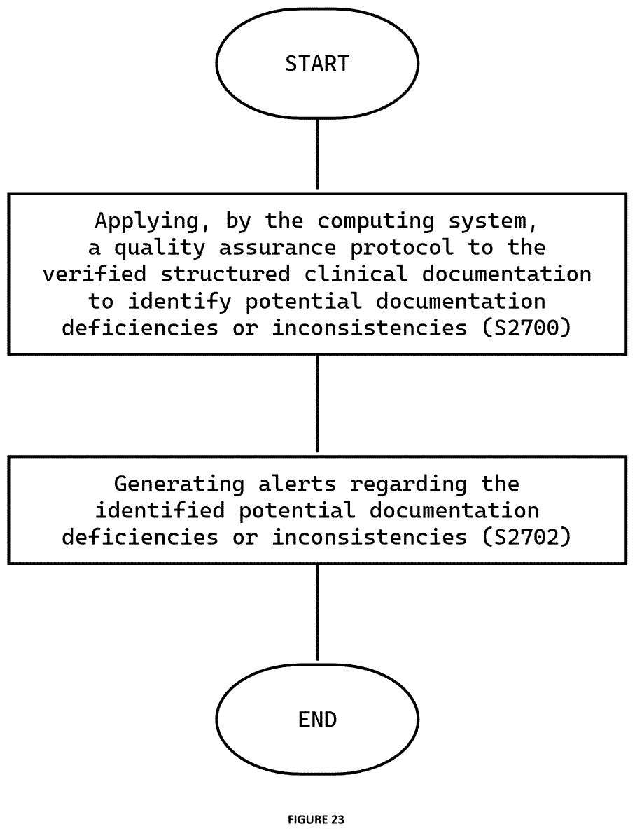

FIG. 23 illustrates the process of applying a quality assurance protocol to verified structured clinical documentation, identifying potential deficiencies or inconsistencies, and generating alerts regarding these issues.

In one embodiment, a computing system is configured to perform a method for automated medical coding. The method begins with the computing system receiving medical information associated with a patient encounter (S100). The received information includes clinical notes, laboratory results, imaging reports, and other relevant data that describe the patient encounter. The computing system then processes the medical information using at least one artificial intelligence model to analyze the content and identify clinical concepts present therein (S102). The analysis involves natural language processing, pattern recognition, and other techniques to extract relevant clinical terminology and entities that are indicative of diagnoses, procedures, or conditions.

Each step in this method is executed sequentially or in parallel depending on system design and implementation. Additionally, various aspects of the method are configured for automation or involve user interaction based on predefined thresholds or conditions. The described embodiments illustrate how the computing system leverages artificial intelligence and standardized coding systems to streamline and enhance the accuracy of medical coding processes.

One implementation initiates by receiving medical information associated with a patient encounter (S100), where the term “medical information” is broadly defined to include at least one of the following: audio recordings of patient-provider conversations, electronic health record (EHR) data, clinical notes, laboratory results, imaging reports, medication lists, and patient demographic information. In embodiments that incorporate audio recordings, the system converts the speech content into text using an automatic speech recognition model (S1500) so that the transcribed text can be processed further to extract clinically relevant entities and associated medical concepts. The EHR data incorporates various components, including but not limited to patient demographics, medication histories, past diagnoses, and other clinical observations that collectively provide a comprehensive view of the patient's medical background. Clinical notes, laboratory results, imaging reports, and medication lists undergo analysis whereby each data element is examined using at least one artificial intelligence model (S102) to identify pertinent clinical concepts. The extracted concepts are then mapped to one or more standardized medical codes (S104), and the method subsequently generates a coding recommendation comprising these codes (S106). The integration of diverse data types from audio recordings, EHR systems, clinical documentation, and diagnostic results ensures that a rich and multifaceted dataset is available for analysis, thereby supporting enhanced accuracy in both coding recommendations and structured clinical documentation generation.

In one embodiment, the at least one artificial intelligence model comprises a natural language processing (NLP) model trained on a corpus of medical terminology, clinical guidelines, and coding standards. The NLP model is designed to process and analyze textual medical data derived from patient records, clinical notes, and other healthcare-related documents. By leveraging machine learning techniques, the model is trained using large, annotated datasets that encompass diverse sources such as medical textbooks, clinical journals, coding manuals including ICD, CPT, and SNOMED, and published clinical guidelines. The training process employs supervised learning methods in which the model learns to recognize and interpret medical terms and phrases in context. Advanced language modeling techniques, including tokenization, embedding generation, and transformer-based architecture, are utilized to capture semantic relationships and contextual nuances within the training corpus. As a result, the NLP model is capable of accurately identifying clinically relevant concepts, detecting ambiguities, and mapping extracted terms to corresponding standardized medical codes. In operation, the NLP model processes input data by first performing linguistic analysis to segment and annotate the text with identified medical entities, and then applying context-sensitive mapping algorithms that reference the clinical guidelines and coding standards from its training dataset. The use of a continuously updated training corpus ensures that the model remains current with emerging medical terminology and evolving clinical practices. Feedback mechanisms provide for periodic retraining or adjustment of model parameters based on user input and verification of coding recommendations, further enhancing model accuracy and reliability in real-world applications. The integration of this NLP model into a broader system enables enhanced automation in the generation of coding recommendations and structured clinical documentation, ultimately contributing to more efficient healthcare data processing and heightened accuracy of patient records.

In an exemplary embodiment, the processing of the extracted clinical concepts includes mapping the identified features to a corresponding coding system, wherein the at least one coding system comprises at least one of International Classification of Diseases (ICD), Current Procedural Terminology (CPT), Healthcare Common Procedure Coding System (HCPCS), and Hierarchical Condition Categories (HCC). In this embodiment, once clinical entities are identified and their relationships determined, the computing system selects a coding system based on contextual parameters and applicable regulatory or organizational standards. The mapping is executed by associating each clinical concept with one or more standardized medical codes corresponding to the selected coding system, thereby facilitating both accurate representation of the patient encounter and consistency in documentation across diverse healthcare applications. The chosen coding system supports the generation of coding recommendations that are subsequently reviewed and accepted, rejected, or modified by the user. Additionally, the integration of such coding systems within the computing system permits updates to the coding recommendation via feedback loops and continuous learning mechanisms, ensuring that the mapping of clinical concepts to codes remains aligned with evolving coding criteria and best practices for clinical documentation and reimbursement.

In an embodiment, the method further comprises integrating the coding recommendation with an electronic health record system. This integration is achieved by establishing a secure data communication interface between the computing system and the electronic health record system (S600). The method additionally comprises automatically populating coding fields in the electronic health record system based on the coding recommendation (S602). In one embodiment, the coding recommendation generated by mapping clinical concepts to standardized medical codes is transmitted to the electronic health record system through a predefined interface protocol, thereby enabling the system to automatically fill corresponding coding fields with the recommended codes. The automatic population of coding fields reduces manual data entry and minimizes potential human error by ensuring that the coding recommendation is directly and accurately integrated into the patient record maintained by the electronic health record system. This integration employs industry-standard communication protocols and data formats to ensure compatibility and reliability across disparate healthcare information systems.

In one embodiment, the computing system further comprises analyzing the verified structured clinical documentation generated based on the extracted clinically relevant information and the associated medical data to identify one or more potential clinical documentation enhancement (CDI) opportunities. In various implementations, the system employs advanced natural language processing techniques in conjunction with one or more artificial intelligence models to assess the completeness, consistency, and accuracy of the clinical documentation. The analysis involves comparing the structured clinical documentation against established clinical documentation guidelines, best practices for comprehensive recording, and provider-specific documentation patterns. The computing system identifies discrepancies, omissions, or inefficiencies that could lead to suboptimal clinical coding outcomes or result in missed opportunities for more effective patient care documentation. When such CDI opportunities are detected, the system generates prompts or alerts displayed to the healthcare provider via the user interface, thereby facilitating a review and potential revision of the clinical records. In some embodiments, these CDI opportunities further integrate with the coding recommendation process, enabling the system to generate supplementary suggestions for documentation enhancements that heighten overall accuracy and support compliance in subsequent coding and billing processes.

In one embodiment, the computing system analyzes medical information by first extracting one or more medical entities from the received data (S700). In some implementations, the system leverages natural language processing techniques to identify key entities, including symptoms, medications, clinical findings, and other patient-specific attributes present within textual or audio input. Following extraction, the system determines relationships between the extracted medical entities (S702). The determination of such relationships includes identifying temporal connections, causal relationships, or associations among various entities, thereby enabling the system to correlate clinical observations and measurements in a meaningful manner. Based on these established relationships, the system then infers one or more diagnoses, procedures, or conditions (S704). This inference process involves comparing the relationships and combinations of identified medical entities against known clinical patterns or guidelines and incorporates machine learning models to enhance accuracy and contextual relevance. Overall, the process bolsters the analytical capabilities of the system by ensuring that the extracted and interrelated data generates clinically pertinent output, supporting subsequent mapping to standardized medical codes for further processing.

In some embodiments, the method further compares the generated coding recommendation against a set of coding guidelines to identify potential coding errors or compliance issues (S800). In this implementation, the coding guidelines are stored in a rules database and encompass clinical coding standards, payer requirements, or regulatory criteria, thereby enabling the computing system to determine whether the recommended standardized medical codes conform to the applicable requirements. When the system identifies discrepancies, mismatches, or other potential coding errors during the comparison, it generates one or more alerts, which are displayed to or otherwise communicated with a user (S802). The alerts provide information related to the identified coding issues, include suggestions for remedial action, require additional verification, or prompt a review by the healthcare provider. In certain embodiments, the generation of alerts occurs in real time or periodically as the patient encounter data is processed, thereby ensuring that the healthcare provider is promptly informed regarding instances of potential noncompliance or coding inaccuracies.

In one embodiment, the computing system is further configured to detect ambiguities in the medical information that impact code assignment (S1000). The system performs an ambiguity detection process in which the received medical information is analyzed to identify portions that are incomplete, conflicting, or insufficiently detailed to support an accurate mapping of clinical concepts to standardized medical codes. For instance, the system evaluates narrative portions of the clinical documentation, discrepancies between recorded data elements, or language that is open to multiple interpretations. Upon detecting such ambiguities, the system generates one or more queries to resolve the identified issues (S1002). These queries are formulated to request additional information, clarification, or confirmation from a healthcare provider or another source to ensure that the subsequent coding recommendation accurately reflects the clinical scenario. The generated queries are presented via the user interface, prompting the user to provide supplemental details or clarifications, thereby reducing the risk of misinterpretation during the coding process.

The method further involves continuous monitoring of the encounter, wherein the computing system periodically updates the structured clinical documentation (S2500, S2502) and displays the updated documentation in real-time (S2504). Additionally, the system applies quality assurance protocols to the verified documentation (S2700) while concurrently generating alerts for any detected potential deficiencies or inconsistencies (S2702) as the patient care session proceeds. Integration with an electronic health record (EHR) system (S600) is executed in real-time, wherein the coding recommendation is automatically populated into the relevant EHR fields (S602). Throughout the patient encounter, user inputs regarding acceptance, rejection, or modification of the real-time coding recommendations are received (S500), and the computing system updates the artificial intelligence model based on the received feedback (S502) to refine subsequent recommendations. This real-time performance ensures that the coding recommendations, confidence scores, and structured clinical documentation remain continuously synchronized with the evolving patient data, thereby enabling timely clinical decision support (S2100, S2102) and enhanced accuracy in clinical documentation and coding.

The computing system further employs a plurality of specialized artificial intelligence models, each specifically configured for a distinct medical specialty. In some embodiments, each specialized model is trained using data sets that incorporate specialty-specific terminologies, clinical guidelines, and coding patterns. For example, one specialized model is optimized for cardiology through exposure to cardiology-specific clinical nomenclature, treatment protocols, and diagnostic criteria, while another model is trained for oncology by leveraging pathology reports, clinical trial guidelines, and tumor staging systems. The system identifies a specialty attribute from the received medical information or associated clinical interaction data and then selects the appropriate model to analyze the data. By employing specialized models, the computing system enhances the precision of clinical concept identification and the mapping process to standardized medical codes, ensuring that the generated coding recommendations are highly relevant to the specific clinical context. This selective application of models further facilitates the generation of structured clinical documentation that reflects the nuanced practices and regulatory requirements unique to each medical specialty.

In some embodiments, the clinical interaction data includes one or more of the following: audio recordings of conversations between a healthcare provider and a patient, video recordings of the patient encounter, real-time transcription of spoken dialogue, handwritten notes, structured form entries, and dictation recordings. At S1300, the computing system receives this clinical interaction data and processes it with one or more artificial intelligence models (S1302) to extract clinically relevant information. The extracted data is subsequently transformed into structured clinical documentation (S1304) that complies with predefined documentation standards.

For example, audio and video recordings provide rich contextual information regarding the patient encounter, while real-time transcriptions generated via advanced speech recognition algorithms (S1500) convert spoken dialogue into text immediately. Handwritten notes, structured form entries, and dictation recordings supplement the comprehensive data set by offering additional perspectives and details not captured solely through audio or video. The integration of these diverse data sources enhances the system's capability to organize and categorize clinical information (S1600, S1602) and to generate natural language narratives for inclusion in the structured documentation (S1604).

By combining information from multiple sources, the system better identifies medical entities, determines relationships between them (S702, S1504), and infers diagnoses, procedures, or conditions (S704). The resulting structured clinical documentation is subsequently reviewed by a healthcare provider (S1306, S1308) and stored in an electronic health record database (S1310), ensuring that the final documentation comprehensively reflects the patient encounter.

By leveraging the fine-tuned large language model, the system is able to identify clinical concepts more accurately and generate mapping recommendations that account for the nuances present in clinical documentation from various medical specialties. This approach also enhances the system's ability to detect ambiguities in the medical information (S1000) and generate queries to resolve such ambiguities (S1002), thereby improving the overall accuracy of the coding recommendation. The inventive system further integrates the coding recommendation with an electronic health record (EHR) system (S600) and automatically populates coding fields in the EHR system based on the coding recommendation (S602).

In one embodiment, processing the clinical interaction data includes converting speech content to text using an automatic speech recognition model. In this embodiment, the computing system receives clinical interaction data, including audio data from the patient encounter, and converts the speech content within such data into a corresponding textual representation. The system employs an automatic speech recognition model that is specifically adapted for clinical environments and is capable of processing complex medical terminologies and variations in speech to produce an accurate transcription. The transcription process is performed in real time or near real time and utilizes language models that have been refined using domain-specific corpora to ensure that the resulting text accurately reflects the clinical dialogue.

Subsequent to identifying clinically relevant entities, the system determines the relationships among these entities by analyzing the syntactic and semantic structures present in the text. The system employs algorithms that infer associations, dependencies, or causal relationships between the extracted entities. For example, it determines that a particular medication is prescribed in response to a diagnosed condition or that certain symptoms are indicative of a specific syndrome. This relationship analysis uses context-based reasoning, dependency parsing, and other natural language understanding methodologies to accurately capture the interconnections among the clinically relevant entities. The resulting network of entities and relationships then supports subsequent operations, such as generating structured clinical documentation or mapping clinical concepts to standardized medical codes.

In one embodiment, generating the structured clinical documentation comprises first organizing the extracted clinically relevant information into predefined sections according to a documentation template (S1600). The extraction process provides a plurality of data elements that are then arranged into corresponding sections of the documentation template based on predefined rules and contextual associations. The organization step ensures that each section is populated by information relevant to that section's intended content, and that the overall structure conforms to the established documentation standards of the healthcare provider system. Subsequent to organizing the information, the method proceeds to populate the documentation template with the organized information (S1602). This step involves mapping and transferring the organized data into the relevant fields and sections of the template, thereby creating a comprehensive and coherently structured clinical document that accurately reflects the patient encounter. Thereafter, the system generates natural language narratives for at least one section of the documentation template (S1604). In this stage, the system employs one or more natural language generation techniques to convert the populated, structured data into descriptive narratives that provide additional context and clarity. The generated narratives are designed to enhance the readability and usability of the documentation by conveying clinically relevant details in a narrative format that can be readily reviewed and interpreted by healthcare providers.

In one embodiment, verifying the structured clinical documentation comprises automatically highlighting portions of the documentation that require particular review by the healthcare provider (S1900). In this embodiment, the computing system identifies sections of the structured clinical documentation that contain ambiguities, non-standard phrases, or potential errors based on predefined criteria and automatically highlights those sections to draw the healthcare provider's attention. The system subsequently receives modifications to the structured clinical documentation from the healthcare provider (S1902). These modifications include corrections, additions, deletions, or reformatting of the highlighted portions using an interactive user interface configured to receive such inputs. After receiving the modifications, the computing system processes the user input and updates the structured clinical documentation based on the modifications (S1904), thereby producing a revised version of the clinical documentation that reflects the accepted corrections and verified information.

In one embodiment, the system is configured to detect unaddressed items (S2200) drawn from a predefined documentation checklist by analyzing extracted clinically relevant information. In this embodiment, the computing system compares the clinically relevant information extracted during the patient encounter with a checklist of required documentation items associated with complete and accurate clinical records. The system identifies discrepancies between the extracted information and the checklist, thereby determining which required documentation items remain unaddressed. Upon detecting one or more unaddressed items, the system generates corresponding alerts (S2202) intended to notify the healthcare provider. These alerts are displayed on a user interface in real time, prompting the healthcare provider to review and address the missing or incomplete documentation. The alerts are generated based on a set of criteria that includes the criticality of the missing information and the potential impact on patient care and compliance with regulatory requirements. Additionally, the system prioritizes or customizes the alerts based on factors such as the patient's condition, the provider's documentation history, or specific practice settings, thereby ensuring that the most relevant unaddressed items are highlighted for immediate review and action.

In some embodiments, the computing system further generates a patient-facing summary of the verified structured clinical documentation in non-technical language. In such embodiments, after the verified structured clinical documentation is stored in the electronic health record database (S1310), the computing system processes the stored documentation to extract key pieces of information relevant and comprehensible to a patient. The system then translates or reformats this technical documentation into a summary designed for easy understanding by a layperson. For example, rather than using medical jargon, the system substitutes simpler language and provides succinct descriptions of diagnoses, treatment recommendations, and other pertinent clinical data. This translation is performed using one or more natural language processing models that are trained to recast clinically precise language into a non-technical summary, preserving the essential content while omitting excessive technical detail. In some implementations, additional user interface elements are provided to allow the healthcare provider to review and, if necessary, manually adjust the patient-facing summary prior to its dissemination. The patient-facing summary is then transmitted to a patient portal or provided directly via a secure user interface, thereby enabling the patient to gain an accessible overview of the clinical information contained in the structured documentation. In embodiments where the computing system is configured to provide real-time updates, the patient-facing summary is dynamically updated as the verified structured clinical documentation is revised during the patient encounter. By generating a patient-facing summary in non-technical language, the system improves patient understanding and facilitates enhanced engagement in shared decision-making regarding their treatment and care.

In one embodiment, after the verified structured clinical documentation is stored in the electronic health record database, the computing system applies a quality assurance protocol to the stored documentation to identify any potential deficiencies or inconsistencies. The quality assurance protocol includes one or more algorithms configured to analyze the content, format, and completeness of the structured clinical documentation and to compare the documentation against predefined documentation standards and regulatory guidelines. For example, the quality assurance protocol automatically evaluates whether all required fields are populated, determines if the clinical narrative is consistent with the associated medical codes, or checks that no essential clinical data elements are missing. When the quality assurance protocol identifies documentation deficiencies or inconsistencies, the computing system generates alerts that communicate the identified issues to the healthcare provider or an appropriate system component. The alerts include details regarding the nature of the deficiency, such as missing information, formatting errors, or discrepancies between clinical findings and documented codes, and prompt the user to review and correct the documentation. Additionally, the system is configured to periodically reapply the quality assurance protocol during a patient encounter or after the documentation is updated, thereby ensuring that any identified deficiencies or inconsistencies are addressed in a timely manner to maintain compliance with coding and clinical documentation standards.

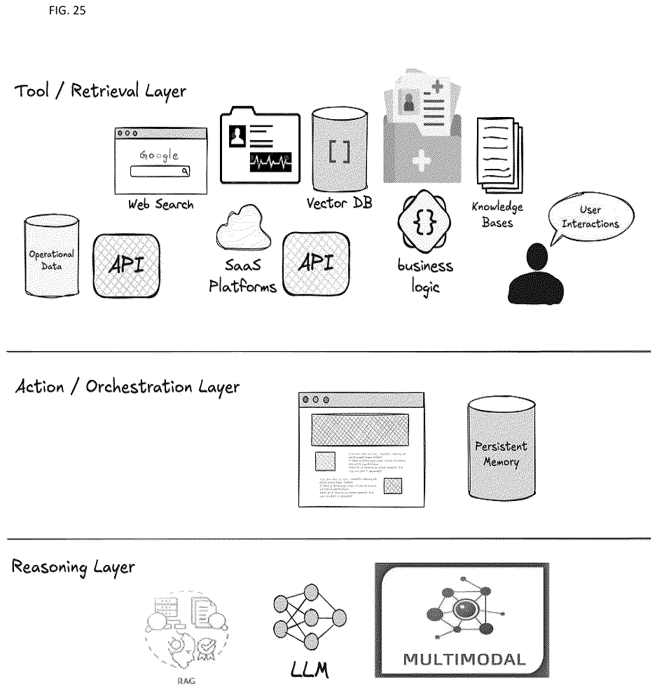

FIGS. 24-25 show exemplary multimodal agentic AI systems. In one case, an encoder-decoder architecture is used for longitudinal health records. The architecture's encoder processes input embeddings to generate hidden representations for each predictor, while the decoder performs cross-attention over these representations to generate diagnostic codes. The system performs sequential generation of codes following clinical priority order-first generating primary diagnoses, then secondary diagnoses based on the primary diagnosis. The encoder-decoder framework proves effective for handling the sequential and hierarchical nature of medical coding.

The method of claim 1, comprising
processing the medical information through a multi-agent agentic artificial intelligence (AI) architecture comprising:
an input agent that extracts and normalizes clinical terminology from unstructured text using natural language processing;
a query agent that maps extracted clinical terms to a medical ontology;
a reasoning agent implementing chain-of-thought processing to simulate clinical decision-making for code selection;
a compliance verification agent that validates coding decisions against current regulatory requirements; and
an orchestration agent that coordinates workflows between the specialized agents;
mapping, by the reasoning agent, the identified clinical concepts to one or more standardized medical codes based on at least one coding system, wherein the reasoning agent employs a large language model (LLM) fine-tuned on medical coding datasets to implement a step-by-step reasoning process that explicitly documents the clinical evidence supporting each code;
generating a coding recommendation comprising the one or more standardized medical codes, wherein the recommendation includes confidence scores indicating the probability that each standardized medical code is appropriate based on the supporting clinical evidence;
validating the coding recommendation through the compliance verification agent to ensure adherence to current coding guidelines, including detecting conflicting or mutually exclusive codes; and
storing the validated standardized medical codes in association with a patient record in a structured format that maintains links to the supporting clinical evidence.
11. The method of claim 1, wherein the input agent comprises a multi-stage natural language processing pipeline that sequentially performs tokenization, part-of-speech tagging, named entity recognition, and dependency parsing on the medical information; a medical entity recognition module trained on annotated clinical corpora to identify domain-specific entities including diagnoses, procedures, medications, laboratory values, and anatomical structures; a contextual modifier detection system that identifies and processes negation, uncertainty, temporality, and experiencer attributes for each extracted clinical concept; and a clinical relationship extraction component that identifies connections between extracted entities, including causal relationships, temporal sequences, and hierarchical associations.
12. The method of claim 1, wherein the query agent comprises a graph traversal algorithm that identifies optimal paths between clinical concepts and relevant medical codes by analyzing node relationships, edge weights, and semantic distances.
13. The method of claim 1, wherein the reasoning agent's chain-of-thought processing:
implements an explicit multi-step reasoning procedure comprising: (i) identifying the principal diagnosis or primary reason for encounter, (ii) detecting relevant comorbidities and complications, (iii) determining procedure justifications, (iv) evaluating coding hierarchy requirements, and (v) applying code sequencing rules;
maintains an evolving belief state represented as a directed acyclic graph (DAG) where nodes represent clinical concepts and edges represent logical relationships between concepts;
applies medical coding guidelines through a rule-based inference engine that references rules from official coding manuals; and
generates uncertainty estimations for each reasoning step using Bayesian confidence scoring.
14. The method of claim 1, wherein the compliance verification agent:
maintains a continuously updated database of coding guidelines, regulatory requirements, National Correct Coding Initiative (NCCI) edits, and payer-specific policies;
implements real-time validation against Medicare's Outpatient Code Editor (OCE) and Medicare Code Editor (MCE) rules to identify medically unlikely edits, gender conflicts, age conflicts, and mutually exclusive code combinations;
calculates a compliance risk score for each code assignment based on historical audit patterns, documentation completeness, and regulatory focus areas; and
validates documentation adequacy by comparing extracted clinical evidence against specialty-specific documentation requirements stored in a structured knowledge base.
15. The method of claim 1, wherein the orchestration agent:
implements an asynchronous message-passing architecture that enables parallel processing across specialized agents while maintaining coordination through a shared blackboard system;
manages processing workflows using a dynamic task scheduling algorithm that optimizes resource allocation based on task complexity, priority, and available computational resources;
employs a timeout and retry mechanism that handles agent processing failures by implementing progressive backoff strategies and fallback processing paths;
maintains a comprehensive execution trace documenting inter-agent communications, decision points, and processing metrics for system auditability; and
adapts processing workflows based on document complexity metrics, automatically adjusting processing depth and agent invocation sequences to optimize performance.
16. The method of claim 1, further comprising integrating with electronic health record (EHR) systems through a FHIR-compliant (Fast Healthcare Interoperability Resources) API that enables standardized data exchange with diverse EHR platforms and real-time document listeners that identify when clinical documentation is finalized and ready for coding.
17. The method of claim 1, wherein processing the medical information further comprises analyzing multimodal clinical data sources including:
processing structured laboratory results through reference range normalization and clinical significance analysis;
extracting relevant findings from diagnostic imaging reports using a specialized image report NLP model;
integrating vital sign measurements and trending analysis to support medical necessity documentation;
correlating medication administration records with documented indications to validate treatment-related codes; and
synchronizing temporal data across multiple clinical sources to establish coherent treatment timelines that support sequential code assignment.

To improve performance, the multi-agent architecture is deployed across a distributed computing environment comprising: a load balancing system that distributes processing tasks across multiple computing nodes based on current utilization, available memory, and processing capabilities; a distributed cache that maintains frequently accessed knowledge graph components, coding rules, and patient context information to minimize redundant data retrieval; and a parallelized processing framework that enables simultaneous analysis of different sections of clinical documentation across multiple processing units;

A Multimodal BEHRT (M-BEHRT) extends transformer capabilities by combining two specialized models: Tabular BEHRT and Text BEHRT. This architecture processes both structured tabular data (clinical features, biological measurements, therapies) and unstructured free-text medical reports simultaneously. The system leverages a pretraining approach similar to BERT's masked language model but applies it to multimodal EHR data. Tabular BEHRT processes structured data from each medical visit, while Text BEHRT handles the corresponding free-text reports. These components are then integrated through a cross-attention module that creates a unified representation of the patient's clinical trajectory.

The system leverages LLMs to autonomously generate and execute code for multi-tabular reasoning within electronic health records, effectively enabling clinicians to interact directly with EHRs using natural language. The following components work together to enable effective autonomous operation:

Medical Information Integration: The system incorporates query-specific medical information to enable effective reasoning based on the given query. This allows Agent to identify and retrieve the necessary tables and records to answer clinical questions.

Demonstration Optimization through Long-Term Memory: Using a long-term memory component, the system maintains successful cases from past experiences and dynamically selects the most relevant few-shot examples to improve performance on new queries.

Interactive Coding with Execution Feedback: Agent implements a multi-turn conversation between the LLM agent (code generator) and code executor, enabling iterative refinement of the generated code-based plan for complex multi-hop reasoning.

Error Tracing and Debugging: Rather than simply returning execution errors, the system thoroughly analyzes error messages to identify underlying causes and iteratively refines solutions until successful completion.

One implementation provides interface agents-LLM-enabled agents that act by controlling user interfaces rather than directly accessing databases through code. These systems can navigate healthcare applications, fill forms, and extract information by interacting with existing graphical interfaces in ways similar to human users. Interface agents involve three core components: interface representation (understanding the structure and elements of the interface), action sequence planning (determining the sequence of interactions needed to accomplish a task), and action execution (carrying out the planned interactions).

A Multi-Agent System is used for Medical Coding. One framework uses five specialized agents, each with distinct roles in the coding process: the patient agent, physician agent, coder agent, reviewer agent, and adjuster agent. This multi-agent system addresses several limitations of traditional LLM approaches to medical coding, particularly the challenges related to the high-dimensional and skewed distribution of ICD codes. By decomposing the complex coding task into specialized roles with defined interactions, the system achieves improved accuracy while maintaining transparency in the decision-making process. The approach integrates with the SOAP (Subjective, Objective, Assessment, and Plan) structure commonly used in electronic health records. Rather than directly generating ICD codes, the system first processes the subjective and objective sections to generate assessment and plan sections, then compares its generation with gold standards to self-correct any hallucinations before final code assignment. The specialized roles within the multi-agent coding system function as follows:

Patient Agent: Reviews service documentation and assigned codes, appealing to the adjuster agent if errors or overcharges are identified.

Physician Agent: Documents observations, interventions, and outcomes in discharge summaries, validates codes generated by the coder agent, and responds to coder queries.

Coder Agent: Analyzes clinical documentation, applies coding guidelines, and assigns appropriate ICD codes based on documented conditions and procedures.

Reviewer Agent: Audits assigned codes for accuracy, compliance with guidelines, and proper documentation support.

Adjuster Agent: Resolves coding disputes between patients, physicians, and coders, making final determinations on contested codes.

This collaborative workflow enables strategic cooperation and contestation among agents to maximize coding accuracy. The confrontational strategy employed between agents further enhances performance by leveraging external knowledge of code descriptions and self-correction mechanisms.

One system uses iterative self-reflection to correct outputs from an LLM, minimizing the need for human editing and evaluation while ensuring both factual accuracy and comprehensibility. The agentic AI bridges the communication gap between clinical terminology and patient understanding.

Reasoning Retrieval-Augmented Generation (Reasoning RAG) workflow, which leverages six LLM-powered agents: retrieval, KG query generation, abstraction, checker, rewrite, and summarization.

In one aspect:
21. A method for electronic health record documentation, comprising:
receiving, by a computing system, clinical interaction data associated with a patient encounter;
processing, by the computing system using at least one artificial intelligence model, the clinical interaction data to extract clinically relevant information;
generating, by the computing system, a structured clinical documentation based on the extracted clinically relevant information, wherein the structured clinical documentation conforms to predefined documentation standards; and presenting, via a user interface, the structured clinical documentation for review by a healthcare provider;
receiving, via the user interface, verification input from the healthcare provider regarding the structured clinical documentation;
storing the verified structured clinical documentation in an electronic health record database.
22. The method of 21, wherein the clinical interaction data comprises at least one of: audio recordings of a conversation between a healthcare provider and a patient, video recordings of the patient encounter, real-time transcription of spoken dialogue, handwritten notes, structured form entries, and dictation recordings.
23. The method of 21, wherein receiving the clinical interaction data comprises capturing, in real-time, audio data of the patient encounter using at least one audio capture device positioned within an examination room.
24. The method of 21, wherein the at least one artificial intelligence model comprises a natural language processing (NLP) model trained on a corpus of medical terminology, clinical guidelines, and documentation standards.
25. The method of 21, wherein the at least one artificial intelligence model comprises a large language model (LLM) fine-tuned on a dataset of annotated clinical documentation from a plurality of medical specialties.
26. The method of 21, wherein processing the clinical interaction data comprises:
converting speech content within the clinical interaction data to text using an automatic speech recognition model;
identifying clinically relevant entities within the text;
determining relationships between the identified clinically relevant entities.
27. The method of 21, wherein generating the structured clinical documentation comprises: organizing the extracted clinically relevant information into predefined sections according to a documentation template;
populating the documentation template with the organized information;
generating natural language narratives for at least one section of the documentation template.
28. The method of 21, wherein the structured clinical documentation comprises at least sections for chief complaint, history of present illness, review of systems, physical examination findings, assessment, and plan.
29. The method of 21, further comprising:
analyzing the extracted clinically relevant information to identify potential gaps or inconsistencies in the clinical documentation;
generating prompts for the healthcare provider to address the identified potential gaps or inconsistencies.
30. The method of 21, further comprising customizing the structured clinical documentation based on at least one of:
the healthcare provider's specialty, the healthcare provider's previous documentation patterns, the healthcare provider's preferences, the patient's condition, and the practice setting.
31. The method of 21, wherein verifying the structured clinical documentation comprises:

highlighting portions of the structured clinical documentation that require particular review by the healthcare provider;
receiving modifications to the structured clinical documentation from the healthcare provider;
updating the structured clinical documentation based on the received modifications.
32. The method of 21, further comprising:
generating, based on the verified structured clinical documentation, suggested medical codes according to at least one standardized coding system;
associating the suggested medical codes with the verified structured clinical documentation in the electronic health record database.
33. The method of 21, further comprising:
analyzing the verified structured clinical documentation to identify clinical decision support opportunities;
presenting clinical decision support recommendations to the healthcare provider based on the identified opportunities.
34. The method of 21, further comprising:
detecting unaddressed items from a predefined documentation checklist based on the extracted clinically relevant information;
generating alerts regarding the unaddressed items for the healthcare provider.
35. The method of 21, further comprising:
determining a context of the patient encounter based on the clinical interaction data;
retrieving, based on the determined context, relevant historical information from the patient's previous electronic health records;
incorporating the relevant historical information into the structured clinical documentation.
36. The method of 21, wherein processing the clinical interaction data further comprises:
identifying social determinants of health mentioned during the patient encounter;
incorporating the identified social determinants of health into the structured clinical documentation.
37. The method of 21, further comprising generating a patient-facing summary of the verified structured clinical documentation in non-technical language.
38. The method of 21, further comprising:
continuously monitoring the clinical interaction data during the patient encounter;
periodically updating the structured clinical documentation as the patient encounter progresses;
displaying the updated structured clinical documentation in real-time during the patient encounter.
39. The method of 21, wherein the verification input from the healthcare provider is received through at least one of:
voice commands, touchscreen gestures, keyboard entries, and digital stylus inputs.
40. The method of 21, further comprising:
applying, by the computing system, a quality assurance protocol to the verified structured clinical documentation to identify potential documentation deficiencies or inconsistencies;
generating alerts regarding the identified potential documentation deficiencies or inconsistencies.

The method of 1, comprising
processing the medical information through a multi-agent agentic artificial intelligence (AI) architecture comprising:
   an input agent that extracts and normalizes clinical terminology from unstructured text using natural language processing;
   a query agent that maps extracted clinical terms to a medical ontology;
   a reasoning agent implementing chain-of-thought processing to simulate clinical decision-making for code selection;
   a compliance verification agent that validates coding decisions against current regulatory requirements; and
   an orchestration agent that coordinates workflows between the specialized agents;
mapping, by the reasoning agent, the identified clinical concepts to one or more standardized medical codes based on at least one coding system, wherein the reasoning agent employs a large language model (LLM) fine-tuned on medical coding datasets to implement a step-by-step reasoning process that explicitly documents the clinical evidence supporting each code;
generating a coding recommendation comprising the one or more standardized medical codes, wherein the recommendation includes confidence scores indicating the probability that each standardized medical code is appropriate based on the supporting clinical evidence;
validating the coding recommendation through the compliance verification agent to ensure adherence to current coding guidelines, including detecting conflicting or mutually exclusive codes; and storing the validated standardized medical codes in association with a patient record in a structured format that maintains links to the supporting clinical evidence.
11. The method of 1, wherein the input agent comprises a multi-stage natural language processing pipeline that sequentially performs tokenization, part-of-speech tagging, named entity recognition, and dependency parsing on the medical information; a medical entity recognition module trained on annotated clinical corpora to identify domain-specific entities including diagnoses, procedures, medications, laboratory values, and anatomical structures; a contextual modifier detection system that identifies and processes negation, uncertainty, temporality, and experiencer attributes for each extracted clinical concept; and a clinical relationship extraction component that identifies connections between extracted entities, including causal relationships, temporal sequences, and hierarchical associations.
12. The method of 1, wherein the query agent comprises a graph traversal algorithm that identifies optimal paths between clinical concepts and relevant medical codes by analyzing node relationships, edge weights, and semantic distances.
13. The method of 1, wherein the reasoning agent's chain-of-thought processing:
   implements an explicit multi-step reasoning procedure comprising: (i) identifying the principal diagnosis or primary reason for encounter, (ii) detecting relevant comorbidities and complications, (iii) determining procedure justifications, (iv) evaluating coding hierarchy requirements, and (v) applying code sequencing rules;
   maintains an evolving belief state represented as a directed acyclic graph (DAG) where nodes represent clinical concepts and edges represent logical relationships between concepts;
   applies medical coding guidelines through a rule-based inference engine that references rules from official coding manuals; and
   generates uncertainty estimations for each reasoning step using Bayesian confidence scoring.
14. The method of 1, wherein the compliance verification agent:
   maintains a continuously updated database of coding guidelines, regulatory requirements, National Correct Coding Initiative (NCCI) edits, and payer-specific policies;
   implements real-time validation against Medicare's Outpatient Code Editor (OCE) and Medicare Code Editor (MCE) rules to identify medically unlikely edits, gender conflicts, age conflicts, and mutually exclusive code combinations;
   calculates a compliance risk score for each code assignment based on historical audit patterns, documentation completeness, and regulatory focus areas; and
   validates documentation adequacy by comparing extracted clinical evidence against specialty-specific documentation requirements stored in a structured knowledge base.
15. The method of 1, wherein the orchestration agent:
   implements an asynchronous message-passing architecture that enables parallel processing across specialized agents while maintaining coordination through a shared blackboard system;
   manages processing workflows using a dynamic task scheduling algorithm that optimizes resource allocation based on task complexity, priority, and available computational resources;
   employs a timeout and retry mechanism that handles agent processing failures by implementing progressive backoff strategies and fallback processing paths;
   maintains a comprehensive execution trace documenting inter-agent communications, decision points, and processing metrics for system auditability; and
   adapts processing workflows based on document complexity metrics, automatically adjusting processing depth and agent invocation sequences to optimize performance.
16. The method of 1, further comprising integrating with electronic health record (EHR) systems through a FHIR-compliant (Fast Healthcare Interoperability Resources) API that enables standardized data exchange with diverse EHR platforms and real-time document listeners that identify when clinical documentation is finalized and ready for coding.
17. The method of 1, wherein processing the medical information further comprises analyzing multimodal clinical data sources including:
   processing structured laboratory results through reference range normalization and clinical significance analysis;
   extracting relevant findings from diagnostic imaging reports using a specialized image report NLP model;
   integrating vital sign measurements and trending analysis to support medical necessity documentation;
   correlating medication administration records with documented indications to validate treatment-related codes; and
   synchronizing temporal data across multiple clinical sources to establish coherent treatment timelines that support sequential code assignment.

To improve performance, the multi-agent architecture is deployed across a distributed computing environment comprising: a load balancing system that distributes processing tasks across multiple computing nodes based on current utilization, available memory, and processing capabilities; a distributed cache that maintains frequently accessed knowledge graph components, coding rules, and patient context information to minimize redundant data retrieval; and a parallelized processing framework that enables simultaneous analysis of different sections of clinical documentation across multiple processing units;

APPENDIX B shows pseudocode for implementing an EHR system enhanced with Agentic AI capabilities. The implementation focuses on four key agent types:
Patient Agent: Engages patients before visits to collect information, set goals, and prepare for appointments.
Documentation Agent: Uses ambient listening to automatically generate clinical documentation and suggest medical codes.
Care Coordination Agent: Analyzes patient data to identify care gaps and generates coordination plans.
Administrative Assistant: Automates administrative tasks like message processing and order management to reduce staff burden.

The system incorporates continuous learning mechanisms to improve performance based on user feedback and operational metrics. It also includes robust security and compliance checks to ensure all agent actions meet regulatory requirements. etailed Operations of Agentic AI Components in EHR Implementation

The implementation of Agentic AI in electronic health record systems represents a paradigm shift from traditional passive AI models to autonomous systems capable of independent decision-making and goal-oriented actions. Unlike conventional AI systems that simply respond to queries or perform predefined tasks, these agents proactively pursue objectives, adapt to changing contexts, and coordinate with human users and other agents to optimize healthcare workflows. The following analysis elaborates on the operational mechanics of the four core agents defined in the pseudocode.

### 1. Patient Agent System: Autonomous Patient Engagement

### Core Operational Model

The Patient Agent operates as an autonomous front-end to the healthcare experience, engaging with patients before, during, and after clinical encounters. Its operations begin with a comprehensive initialization process that establishes the agent's context and goals:

```
FUNCTION initialize(patientProfile, upcomingAppointment,
EHRDatabase):
  this.patientProfile = patientProfile
  this.appointmentContext = upcomingAppointment
  this.EHRDatabase = EHRDatabase
  this.conversationHistory = new ConversationBuffer( )
  this.agentGoals = [
    “collect_visit_goals”,
    “verify_demographics”,
    “assess_symptoms”,
    “check_medication_adherence”,
    “identify_social_determinants”
  ]
  this.LLM = loadMedicalLanguageModel(“healthcare-agent-v3”)
```

### Contextual Awareness and Goal-Oriented Behavior

The agent's core autonomous capabilities emerge from its contextual awareness and goal-driven approach to patient interactions. When conducting pre-visit preparation, the agent:
1. Analyzes Appointment Context: The agent determines the nature of the upcoming appointment (e.g., wellness check, specialist consultation, follow-up) and calibrates its interaction strategy accordingly. For a cardiology appointment, the agent would focus on cardiac-relevant symptoms and medication adherence, while a wellness visit might trigger broader health assessment questions.
2. Executes Goal-Directed Conversation: Rather than following a rigid script, the agent dynamically pursues its assigned goals through adaptive questioning:

```
FOREACH goal IN this.agentGoals:
  // Dynamically adjust questioning based on previous responses
  relevantQuestions = filterQuestionsByGoal(questionSet, goal)
  responses = askQuestionsAndProcessResponses(relevantQuestions)
  this.conversationHistory.append(responses)
  // Analyze if goal has been adequately addressed
  IF NOT isGoalFulfilled(goal, responses):
    followUpQuestions = generateFollowUpQuestions(goal, responses)
    additionalResponses =
    askQuestionsAndProcessResponses(followUpQuestions)
    this.conversationHistory.append(additionalResponses)
```

This approach enables the agent to adaptively pursue information until each goal is satisfied. For example, when assessing symptoms, if a patient mentions occasional shortness of breath, the agent would autonomously generate follow-up questions about triggers, duration, and associated symptoms before determining whether the goal of symptom assessment has been adequately fulfilled.

### Clinical Integration Operations

The agent bridges the gap between patient-provided information and clinical workflows through several key operations:
1. Structured Data Extraction: The agent transforms conversational responses into structured clinical data points that can be integrated into the EHR.
2. Visit Summary Generation: After gathering information, the agent synthesizes a concise yet comprehensive pre-visit summary that highlights key issues, changes since last visit, and patient goals.
3. Clinical Team Notification: The agent autonomously determines when information warrants immediate clinical attention and notifies the appropriate team members.
4. Continuous Engagement Loop: Unlike traditional intake forms, the agent maintains an ongoing conversation, adapting to new information and responding to patient questions throughout the process.

The Patient Agent's operations require several capabilities:
1. Contextual Conversation Memory: The ConversationBuffer must maintain not just a record of exchanges but an understanding of the conversation's progression toward defined goals.
2. Domain-Specific Language Understanding: The loadMedicalLanguageModel function loads specialized language models trained on healthcare conversations, ensuring accurate interpretation of medical terminology and symptom descriptions.
3. Multi-turn Dialogue Management: The agent must maintain coherence across multiple conversational turns while pursuing its information-gathering goals.
4. Privacy-Preserving Communication: All operations must maintain strict HIPAA compliance, with appropriate encryption and access controls.

### 2. Clinical Documentation Agent: Ambient Intelligence and Documentation Automation

### Core Operational Model

The Clinical Documentation Agent functions as an ambient intelligence system that transforms clinical encounters into structured documentation while minimizing provider administrative burden. Its initialization establishes provider-specific parameters that guide its documentation approach:
FUNCTION initialize (providerProfile, EHRDatabase, codingSystem):
   this.providerProfile=providerProfile
   this.EHR Database=EHRDatabase
   this.codingSystem=codingSystem
   this.documentationTemplates=loadSpecialtyTemplates(providerProfile.specialty)
this.providerStyleModel=loadOrCreateProviderStyleModel(providerProfile.id)
this.LLM=loadMedicalDocumentationModel(“clinical-documentation-v2”)
this.audioProcessor=initializeAudioProcessor( )
this.medicalKnowledgeBase=connectToKnowledgeBase( )

### Ambient Intelligence Operations

The agent's core documentation capabilities are powered by its ability to process ambient conversations while distinguishing clinically relevant information from extraneous dialogue:
1. Continuous Ambient Monitoring: The agent actively processes the audio stream of clinical encounters, breaking it into manageable segments:

```
WHILE session is active:
  // Process audio in real-time
  audioSegment = this.audioProcessor.captureSegment(30) // 30
  seconds of audio
  // Convert speech to text with speaker identification
  transcribedSegment = transcribeAudioWithSpeakers(audioSegment)
```

2. Speaker Differentiation: The transcribeAudioWithSpeakers function distinguishes between provider and patient voices, essential for accurate attribution of information.
3. Clinical Concept Extraction: Beyond simple transcription, the agent identifies clinically relevant concepts mentioned in the conversation:

// Extract clinical concepts
clinicalConcepts = extractClinicalConcepts(transcribedSegment,
this.medicalKnowledgeBase)

This operation leverages the connected medical knowledge base to recognize mentions of symptoms, medications, procedures, and other clinical entities, even when expressed in colloquial terms.

### Autonomous Documentation Construction

The agent's most complex operations involve transforming extracted clinical concepts into coherent, structured documentation:
1. Dynamic Section Mapping: The agent autonomously determines which sections of the clinical note should contain particular information:
FOREACH concept IN clinicalConcepts:
relevantSection=determineRelevantSection (concept, documentationSession.note_sections) update NoteSection (documentationSession, relevantSection, concept)

This operation requires sophisticated understanding of clinical documentation conventions. For example, a mention of “trouble sleeping” during the review of a medication would be mapped to the medication side effects section rather than the review of systems section.
2. Progressive Note Building: Rather than generating documentation only at the end of an encounter, the agent continuously updates the note as new information emerges, allowing providers to view documentation in real-time.
3. Narrative Synthesis: For each section, the agent transforms discrete clinical concepts into coherent narrative text that matches the provider's documentation style, as captured in the providerStyleModel.

### Medical Coding Operations

The agent extends its capabilities beyond documentation to include autonomous medical coding:

```
FUNCTION generateMedicalCodes(completeDocumentation):
  // Extract diagnoses, procedures, and other billable elements
  clinicalElements = extractBillableElements(completeDocumentation)
  // Generate appropriate medical codes
  suggestedCodes = [ ]
  FOREACH element IN clinicalElements:
    relevantCodes = this.codingSystem.findRelevantCodes(element)
    codingJustification = generateCodingJustification(element,
    relevantCodes)
    suggestedCodes.append({
      “element”: element,
      “codes”: relevantCodes,
      “confidence”: calculateConfidenceScore(element, relevantCodes),
      “justification”: codingJustification
    })
```

This process involves several sophisticated operations:
1. Billable Element Identification: The agent distinguishes between clinical information that warrants coding (diagnoses, procedures, etc.) and contextual information.
2. Code Selection: For each billable element, the agent consults the coding system to identify appropriate codes, considering code specificity, hierarchical relationships, and coding guidelines.
3. Justification Generation: Critically, the agent generates explicit justifications for each suggested code, creating an audit trail and allowing providers to verify coding decisions.
4. Confidence Scoring: The agent quantifies its certainty about each code recommendation, allowing for appropriate human review of uncertain cases.

### Technical Implementation Considerations

The Documentation Agent requires several advanced technical capabilities:
1. High-Accuracy Audio Processing: The ambient listening functionality demands sophisticated audio processing to handle overlapping speech, background noise, and variable acoustic environments.
2. Provider Style Modeling: The loadOrCreateProviderStyleModel function establishes a model of the provider's documentation preferences, including terminology choices, narrative structure, and level of detail.
3. Real-time Processing Pipeline: The entire system must operate with minimal latency to provide value during the clinical encounter rather than only afterward.
4. Contextual Understanding: The agent must understand the broader context of the clinical encounter to accurately interpret and document information.

### 3. Care Coordination Agent: Proactive Care Planning and Resource Optimization Core Operational Model

The Care Coordination Agent functions as an autonomous system for identifying care needs, coordinating resources, and ensuring comprehensive follow-up. Its initialization establishes the organizational context in which it operates:
FUNCTION initialize (healthSystem, EHRDatabase, departmentMappings):
   this.healthSystem=healthSystem
   this.EHRDatabase=EHRDatabase
   this.departmentMappings=departmentMappings
   this.referralRules=loadReferralRules(healthSystem)
   this.resourceAvailability=initializeResourceTracker( )
   this.LLM=loadCoordinationModel “care-coordination-v1”)

### Patient Needs Analysis Operations

The agent begins its operations by analyzing comprehensive patient data to identify care needs:

```
FUNCTION analyzeCareNeeds(patientId, currentEncounterData):
  patient = this.EHRDatabase.getPatientById(patientId)
  // Analyze current and historical data
  patientRiskProfile = generateRiskProfile(patient, currentEncounterData)
  careGaps = identifyCareGaps(patient, patientRiskProfile)
```

This process involves several sophisticated operations:
1. Risk Profile Generation: The agent synthesizes current and historical patient data to create a comprehensive risk profile, considering clinical factors (diagnoses, lab values), demographic factors (age, gender), and social determinants of health.
2. Care Gap Identification: Using the risk profile, the agent identifies specific gaps in the patient's care, such as overdue preventive screenings, unaddressed chronic condition management needs, or follow-up requirements for recent findings.
3. Action Recommendation: For each identified gap, the agent autonomously determines appropriate interventions:

```
FOREACH gap IN careGaps:
  recommendedActions = determineRecommendedActions(gap,
patientRiskProfile)
  careRecommendations.append({
    “gap”: gap,
    “actions”: recommendedActions,
    “priority”: calculatePriority(gap, patientRiskProfile),
    “rationale”: generateClinicalRationale(gap, recommendedActions)
  })
```

4. Prioritization Logic: The agent assigns priority levels to each recommendation based on clinical urgency, patient risk factors, and potential impact on outcomes.

### Resource Coordination Operations

The most distinctive capability of the Care Coordination Agent is its autonomous management of healthcare resources to fulfill identified needs:

```
FUNCTION generateCareCoordinationPlan(patientId,
careRecommendations):
  patient = this.EHRDatabase.getPatientById(patientId)
  // Create coordination plan
  coordinationPlan = {
    “patient”: patient,
    “timestamp”: getCurrentTimestamp( ),
    “care_steps”: [ ]
  }
  FOREACH recommendation IN careRecommendations:
    FOREACH action IN recommendation.actions:
      // Determine required resources
      requiredResources = identifyRequiredResources(action)
      // Check resource availability
      availableTimeSlots =
      this.resourceAvailability.findAvailableSlots(requiredResources)
      // Generate step in care plan
      careStep = {
        “action”: action,
        “department”: determineDepartment(action,
        this.departmentMappings),
        “scheduling_options”: availableTimeSlots,
        “step_owner”: assignStepOwner(action, this.healthSystem),
        “dependencies”: identifyDependencies(action,
        coordinationPlan.care_steps),
        “status”: “scheduled”
      }
```

These operations involve complex decision-making processes:
1. Resource Requirement Analysis: The agent determines what specific resources (provider types, equipment, facilities) are needed for each recommended action.
2. Availability Checking: The agent interfaces with scheduling systems to identify available time slots that match the required resources.
3. Department Mapping: Using organizational knowledge encoded in departmentMappings, the agent determines which specific departments should address each action.
4. Responsibility Assignment: The agent assigns specific ownership of each care step to appropriate roles within the organization.
5. Dependency Management: Critically, the agent identifies dependencies between care steps (e.g., lab work must precede specialist consultation) and orders them accordingly.

### Clinical Validation Operations

To ensure clinical appropriateness, the agent performs validation operations:
//Validate plan for clinical appropriateness
validatePlanAppropriateness (coordinationPlan, patient)

This validation process involves:
1. Guidelines Compliance: Checking that the care plan aligns with clinical practice guidelines for the patient's conditions.
2. Contraindication Screening: Identifying potential contraindications between recommended actions.
3. Patient-Specific Considerations: Accounting for patient preferences, barriers to care, and other individualized factors.

### Technical Implementation Considerations

The Care Coordination Agent requires several advanced technical capabilities:
1. Multi-system Integration: The agent must interface with scheduling systems, staff directories, and departmental resources across the healthcare organization.
2. Clinical Rules Engine: The referralRules and other clinical logic must be encoded in a flexible, maintainable format that can be updated as guidelines change.
3. Resource Modeling: The resourceAvailability tracker must maintain an accurate model of available resources across the organization.
4. Constraint Satisfaction Problem Solving: Finding optimal schedules that meet all dependencies and resource requirements is essentially a constraint satisfaction problem requiring sophisticated algorithms.

### 4. Administrative Assistant: Autonomous Workflow Management Core Operational Model

The Administrative Assistant agent functions as an autonomous system for managing the numerous administrative tasks that surround clinical care. Its initialization establishes the context and rules for administrative actions:
FUNCTION initialize(userProfile, EHRSystem, workflowRules):
this.userProfile=userProfile
this.EHRSystem=EHRSystem
this.workflowRules=workflowRules
this.taskQueue=PriorityQueue( )
this.LLM=loadAdministrativeModel(“healthcare-admin-v2”)
this.userPreferences=loadUserPreferences(userProfile.id)

### Task Monitoring and Prioritization Operations

The agent continuously monitors incoming tasks from various sources, prioritizing them based on urgency, importance, and user preferences:

```
FUNCTION monitorIncomingTasks( ):
  WHILE system is active:
    // Check for new tasks from various sources
    inboxMessages =
    this.EHRSystem.checkForNewMessages(this.userProfile.id)
    pendingOrders =
    this.EHRSystem.getPendingOrders(this.userProfile.id)
    documentationTasks =
    this.EHRSystem.getDocumentationTasks(this.userProfile.id)
    // Process and prioritize tasks
    FOREACH message IN inboxMessages:
      prioritizedTask =
      classifyAndPrioritizeTask(message, “message”)
      this.taskQueue.add(prioritizedTask)
```

This operation involves several sophisticated processes:
1. Multi-source Monitoring: The agent actively polls multiple systems for new tasks requiring attention.
2. Task Classification: Each incoming item is classified based on content, urgency, and type, using natural language processing to understand the nature of messages and requests.
3. Dynamic Prioritization: The agent applies complex prioritization rules that consider clinical urgency, organizational policies, and user preferences to determine the optimal order for task processing.

### Autonomous Task Processing

The agent's most advanced capabilities involve determining the appropriate level of automation for each task and taking action accordingly:

```
FUNCTION processHighPriorityTasks( ):
  WHILE this.taskQueue is not empty:
    task = this.taskQueue.getHighestPriority( )
    // Determine if task can be automated
    automationLevel = determineAutomationLevel(task,
    this.workflowRules)
    IF automationLevel == “fully_automated”:
      // Handle task without human intervention
      processAutomatedTask(task)
      recordTaskCompletion(task, “automated”)
    ELSE IF automationLevel == “semi_automated”:
      // Prepare draft response or action
      draftAction = prepareDraftAction(task)
      // Present to user for verification
      userVerification = presentForVerification(draftAction,
      this.userProfile)
      IF userVerification.approved:
        completeTask(task, draftAction, userVerification.modifications)
        recordTaskCompletion(task, “semi_automated”)
      ELSE:
        rescheduleTask(task)
```

This implementation demonstrates several key autonomous capabilities:
1. Automation Level Assessment: The agent applies organization-specific rules to determine whether a task can be fully automated, partially automated, or requires complete human attention.
2. Fully Autonomous Actions: For appropriate tasks, the agent takes action without human intervention, such as routing routine lab results to the correct department or scheduling standard follow-ups.
3. Semi-Autonomous Draft Creation: For tasks requiring human oversight, the agent prepares draft responses or actions for human verification.
4. Verification Interface: The agent presents draft actions to users in an efficient interface that minimizes review time while ensuring appropriate oversight.

### Context-Aware Response Generation

For communication tasks, the agent generates context-aware responses by gathering relevant information:

```
IF taskType == “message”:
  // Generate draft response
  messageContent = task.content
  relevantContext = gatherRelevantContext(task.patientId,
  messageContent)
  draftResponse = this.LLM.generateResponse(messageContent,
relevantContext, this.userPreferences)
```

This process involves:
1. Context Gathering: The agent retrieves relevant patient information, previous communications, and applicable policies that inform an appropriate response.
2. Personalized Response Generation: Using the loaded language model, the agent generates responses that match the user's communication style and preferences.
3. Compliance Checking: The agent ensures all communications comply with organizational policies and regulatory requirements.

The Administrative Assistant agent requires several technical capabilities:
1. Task Classification System: Sophisticated natural language understanding to classify the nature and urgency of incoming tasks.
2. Priority Queue Management: Advanced data structures to maintain an optimally ordered task list that can be dynamically updated as new tasks arrive.
3. Context Retrieval System: Efficient retrieval of relevant information from across the EHR and other systems to inform task processing.
4. User Preference Modeling: Systems to learn and adapt to individual users' working preferences and communication styles.

The four agents do not operate in isolation but function as an integrated system with sophisticated coordination mechanisms. The AgenticEHRSystem class orchestrates their operations:

```
FUNCTION handlePatientEncounter(patientId, providerId, encounterType):
  patient = this.patientDatabase.getPatientById(patientId)
  provider = this.users.getProviderById(providerId)
  // Pre-visit preparation
  preVisitData = this.patientAgent.conductPreVisitPreparation(patient, encounterType)
  // Initialize documentation session
  documentationSession = this.documentationAgent.beginEncounterDocumentation(patientId, encounterType)
  // Real-time documentation
  completeDocumentation = this.documentationAgent.processAmbientConversation(documentationSession)
  // Generate medical codes
  suggestedCodes = this.documentationAgent.generateMedicalCodes(completeDocumentation)
  // Generate care coordination plan
  careRecommendations = this.careCoordinationAgent.analyzeCareNeeds(patientId, completeDocumentation)
  coordinationPlan = this.careCoordinationAgent.generateCareCoordinationPlan(patientId,
careRecommendations)
```

This orchestration involves several key integration points:
1. Sequential Workflow Triggering: The system triggers agent actions in a logical sequence that mirrors the patient care journey.
2. Data Sharing Between Agents: Information gathered by the Patient Agent informs the Documentation Agent's work, which in turn provides input to the Care Coordination Agent.
3. Cross-Agent Learning: Feedback collected on one agent's performance can inform improvements across the entire system.
4. Unified Security and Compliance Framework: All agents operate within a common security and compliance framework managed by the SecurityAndComplianceManager.

The detailed operations of these four agentic AI components represent a shift in how EHR systems function. Rather than passive data repositories requiring extensive human operation, these agents transform the EHR into an active participant in the healthcare process, autonomously managing routine tasks, identifying care needs, and supporting clinical decision-making.

This implementation aligns with the healthcare industry's pressing need to reduce administrative burden, improve care coordination, and enhance the patient experience. By encoding clinical knowledge, organizational policies, and user preferences into autonomous agents that can take independent action when appropriate, this system addresses many of the fundamental challenges facing modern healthcare delivery.

1. A method for an automated classification of information by a computer, comprising:
applying at least one agentic artificial intelligence (AI) model including a Chain of Thought (CoT) large language model (LLM) with a domain-specific attention backtracking and when a conflict is encountered the AI model reconsiders earlier reasoning if a later determination invalidates a previous conclusion;
performing a multi-stage time-dependent reasoning with a context-aware tokenizer that preserves terminology relationships and hierarchies; and
maintaining explicit documentation linkage, creating a citation for each classification to specific evidence in the information, wherein the AI model implements a multi-step process that applies specificity refinement by searching for details that support a specific classification.

2. The method of claim 1, wherein the information comprises at least one of: audio recordings of patient-provider conversations, electronic health record (EHR) data, clinical notes, laboratory results, imaging reports, medication lists, and patient demographic information, comprising analyzing the medical information comprises: extracting medical entities from the medical information; determining relationships between the extracted medical entities; and inferring diagnoses, procedures, or conditions based on the determined relationships.

3. The method of claim 1, wherein the artificial intelligence model comprises a natural language processing (NLP) model trained on a corpus of medical terminology, clinical guidelines, and coding standards, and wherein different specialized artificial intelligence models are provided for different medical specialties, with each specialized artificial intelligence model trained on specialty-specific terminology, guidelines, and coding patterns.

4. The method of claim 1, wherein the automated classification is based on at least one of: International Classification of Diseases (ICD), Current Procedural Terminology (CPT), Healthcare Common Procedure Coding System (HCPCS), and Hierarchical Condition Categories (HCC).

5. The method of claim 1, further comprising: determining, for each standardized, a confidence score indicating a probability that the standardized medical code is appropriate; and displaying a reason and a confidence score in association with each standardized code.

6. The method of claim 5, further comprising displaying a summary of relevant clinical evidence from the medical information that supports each recommended medical code.

7. The method of claim 1, further comprising detecting a potential missing documentation required for complete and accurate coding; and generating alerts regarding the potential missing documentation.

8. The method of claim 1, further comprising identifying one or more potential clinical documentation improvement (CDI) opportunities based on the information and generating suggestions for additional documentation.

9. The method of claim 1, further comprising:
integrating with an electronic health record (EHR) system; and automatically populating coding fields in the EHR system;
processing with the artificial intelligence model, clinical interaction data to extract clinically relevant information; and
generating a structured clinical documentation based on the extracted clinically relevant information, wherein the structured clinical documentation conforms to predefined documentation standards.

10. (canceled)

11. A system, comprising:
a processor configured to perform:
multi-stage natural language processing pipeline that sequentially performs tokenization, part-of-speech tagging, named entity recognition, and dependency parsing on information;
entity recognition trained on annotated corpora to identify domain-specific entities;
contextual modifier detection that identifies and processes negation, uncertainty, temporality, and attributes for extracted concepts; and
relationship extraction that identifies connections between extracted entities, including causal relationships, temporal sequences, and hierarchical associations.

12. The system of claim 11, comprising a query agent-with a graph traversal algorithm that identifies optimal paths between concepts and codes by analyzing node relationships, edge weights, and semantic distances.

13. The system of claim 11, comprising a reasoning agent with code that:
implements an explicit multi-step reasoning procedure including: (i) identifying a diagnosis or reason for encounter, (ii) detecting relevant comorbidities and complications, (iii) determining procedure justifications, (iv) evaluating coding hierarchy requirements, and (v) applying code sequencing rules;
maintains an evolving belief state represented as a directed acyclic graph (DAG) where nodes represent clinical concepts and edges represent logical relationships between concepts;
applies medical coding guidelines through a rule-based inference engine that references rules from official coding manuals; and
generates uncertainty estimations for each reasoning step using Bayesian confidence scoring.

14. The system of claim 11, comprising a compliance verification agent that:
maintains a continuously updated database of coding guidelines, regulatory requirements, National Correct Coding Initiative (NCCI) edits, and payer-specific policies;
implements real-time validation against Medicare's Outpatient Code Editor (OCE) and Medicare Code Editor (MCE) rules to identify medically unlikely edits, gender conflicts, age conflicts, and mutually exclusive code combinations;
calculates a compliance risk score for each code assignment based on historical audit patterns, documentation completeness, and regulatory focus areas; and
validates documentation adequacy by comparing extracted clinical evidence against specialty-specific documentation requirements stored in a structured knowledge base.

15. The system of claim 11, comprising an orchestration agent that:
implements an asynchronous message-passing architecture that enables parallel processing across specialized agents while maintaining coordination through a shared blackboard system;
manages processing workflows using a dynamic task scheduling algorithm that optimizes resource allocation based on task complexity, priority, and available computational resources;
employs a timeout and retry mechanism that handles agent processing failures by implementing progressive backoff strategies and fallback processing paths;
maintains a comprehensive execution trace documenting inter-agent communications, decision points, and processing metrics for system auditability; and
adapts processing workflows based on document complexity metrics, automatically adjusting processing depth and agent invocation sequences to optimize performance.

16. The method of claim 1, further comprising integrating with electronic health record (EHR) systems through a FHIR-compliant (Fast Healthcare Interoperability Resources) API that enables standardized data exchange with diverse EHR platforms and real-time document listeners that identify when clinical documentation is finalized and ready for coding.

17. The method of claim 1, further comprising:
processing structured laboratory results through reference range normalization and clinical significance analysis;
extracting relevant findings from diagnostic imaging reports using a specialized image report NLP model;
integrating vital sign measurements and trending analysis to support medical necessity documentation;
correlating medication administration records with documented indications to validate treatment-related codes; and
synchronizing temporal data across multiple clinical sources to establish coherent treatment timelines that support sequential code assignment.

18. A multi-agent agentic artificial intelligence (AI) architecture comprising:
an input agent that extracts and normalizes terminology from unstructured text using natural language processing;
a query agent that maps extracted terms to an ontology;
a reasoning agent implementing chain-of-thought processing for code selection;
a compliance verification agent that validates coding decisions; and
an orchestration agent that coordinates workflows between the specialized agents; and
an agentic AI model to perform:
mapping, by the reasoning agent, the identified concepts to one or more standardized codes based on at least one coding system, wherein the reasoning agent employs a large language model (LLM) fine-tuned on coding datasets to implement a step-by-step reasoning process that explicitly documents the evidence supporting each code;
generating a coding recommendation comprising the one or more standardized medical codes, wherein the recommendation includes confidence scores indicating the probability that each standardized code is appropriate based on evidence;
validating the coding recommendation through the compliance verification agent to ensure adherence to coding guidelines, including detecting conflicting or mutually exclusive codes; and
storing the validated standardized codes in a structured format that maintains links to supporting evidence.

19. The architecture of claim 18, wherein the agentic AI model receives a natural language request in a first language, processes the request in a second language, and responds back in the first language.

20. (canceled)

21. The architecture of claim 18, wherein the agentic AI model polls multiple systems for tasks requiring attention, each incoming item is classified based on content, urgency, and type, using natural language processing to understand messages and requests, wherein the AI model dynamically prioritizes tasks considering urgency, organizational policies, and user preference to determine an order for task processing.

22. The system of claim 11, comprising:
an input agent that extracts and normalizes terminology from unstructured text using natural language processing;
a query agent that maps extracted terms to an ontology;
a reasoning agent implementing chain-of-thought processing to simulate decision-making for code selection;
a compliance verification agent that validates coding decisions
an orchestration agent that coordinates workflows between specialized agents; and
an AI model for:
mapping, by the reasoning agent, concepts to one or more standardized codes to implement a step-by-step reasoning process supporting each code;
generating a coding recommendation comprising the one or more standardized codes, wherein the recommendation includes confidence scores indicating the probability that the standardized code is appropriate based on the evidence;
validating the coding recommendation through the compliance verification agent to ensure adherence to coding guidelines, including detecting conflicting or mutually exclusive codes; and
storing the validated standardized codes in a structured format that maintains links to the evidence.
